(19) United States
(12) Patent Application Publication
(10) Pub. No.: US 2026/0042779 A1
(43) Pub. Date: Feb. 12, 2026

(54) SYNTHESIS OF SUBSTITUTED 1-ARYL-1'-HETEROARYL COMPOUNDS AND SUBSTITUTED 1,1'-BIHETEROARYL COMPOUNDS, AND ANALOGUES THEREOF

(71) Applicant: Arbutus Biopharma Corporation, Vancouver (CA)

(72) Inventors: Andrew G. Cole, Cranbury, NJ (US); Jeremy Mason, Warminster, PA (US); Duyan Nguyen, Ambler, PA (US); Seyma Ozturk, Southampton, PA (US); Mahesh Kumar Pallerla, Chalfont, PA (US); Ganapati Reddy Pamulapati, Warrington, PA (US); Jan Michelle Spink, Hatboro, PA (US)

(73) Assignee: Arbutus Biopharma Corporation, Vancouver, BC (CA)

(21) Appl. No.: 19/099,291

(22) PCT Filed: Aug. 9, 2023
(86) PCT No.: PCT/IB2023/058055

§ 371 Date: Jan. 28, 2025

Related U.S. Application Data
(60) Provisional application No. 63/398,452, filed on Aug. 16, 2022.

(51) Int. Cl. C07D 519/00; A61K 31/444; C07D 213/64; C07F 5/02

(52) U.S. Cl. CPC C07D 519/00; A61K 31/444; C07D 213/64; C07F 5/025

(57) ABSTRACT

The present disclosure includes synthetic methods for preparing certain substituted 1-aryl-1′-heteroaryl and 1,1′-biheteroaryl compounds, which can be used to treat, ameliorate, and/or prevent hepatitis B virus (HBV) infections in a patient.

### CROSS-REFERENCE TO RELATED APPLICATIONS

The present application claims priority under 35 U.S.C. § 119(e) to U.S. Provisional Patent Application No. 63/398,452, filed Aug. 16, 2022, which is incorporated herein by reference in its entirety.

### BACKGROUND

Hepatitis B virus (HBV) is a noncytopathic, liver tropic DNA virus belonging to Hepadnaviridae family. HBV infection is one of the world's most prevalent diseases, being listed by National Institute of Allergy and Infectious Diseases (NIAID) as a High Priority Area of Interest. Although most individuals resolve the infection following acute symptoms, approximately 30% of cases become chronic. 350-400 million people worldwide are estimated to have chronic hepatitis B, leading to 0.5-1 million deaths per year, due largely to the development of hepatocellular carcinoma, cirrhosis and/or other complications.

A limited number of drugs are currently approved for the management of chronic hepatitis B, including two formulations of alpha-interferon (standard and pegylated) and five nucleoside/nucleotide analogues (lamivudine, adefovir, entecavir, telbivudine, and tenofovir) that inhibit HBV DNA polymerase. At present, the first-line treatment choices are entecavir, tenofovir and/or peg-interferon alfa-2a. However, peg-interferon alfa-2a achieves desirable serological milestones in only one third of treated patients, and is frequently associated with severe side effects. Entecavir and tenofovir are potent HBV inhibitors, but require long-term or possibly lifetime administration to continuously suppress HBV replication, and may eventually fail due to emergence of drug-resistant viruses. There is thus a pressing need for the introduction of novel, safe and effective therapies for chronic hepatitis B.

Hepatitis D virus (HDV) is a small circular enveloped RNA virus that can propagate only in the presence of HBV. In particular, HDV requires the HBV surface antigen protein to propagate itself. Infection with both HBV and HDV results in more severe complications compared to infection with HBV alone. These complications include a greater likelihood of experiencing liver failure in acute infections and a rapid progression to liver cirrhosis, with an increased chance of developing liver cancer in chronic infections. In combination with hepatitis B virus, hepatitis D has the highest mortality rate of all the hepatitis infections. The routes of transmission of HDV are similar to those for HBV. Infection is largely restricted to persons at high risk of HBV infection, particularly injecting drug users and persons receiving clotting factor concentrates.

Currently, there is no effective antiviral therapy available for the treatment of acute or chronic type D hepatitis. Interferon-alfa, given weekly for 12 to 18 months, is the only licensed treatment for hepatitis D. Response to this therapy is limited-in only about one-quarter of patients is serum HDV RNA undetectable 6 months post therapy.

There is thus a need in the art for novel compounds and/or compositions that can be used to treat, ameliorate, and/or prevent HBV infection in a subject. In certain embodiments, the compounds can be used in patients that are HBV infected, patients who are at risk of becoming HBV infected, and/or patients that are infected with drug-resistant HBV. In other embodiments, the HBV-infected subject is further HDV-infected. There is further a need in the art to identify scalable synthetic schemes that allow for the preparation of large scale batches of such compounds. The present disclosure addresses this need.

### BRIEF SUMMARY

The present disclosure provides methods of preparing 2-((6-(2-chloro-3-(3-chloro-2-(3-methoxy-4-((7-oxo-2,6-diazaspiro[3.4]octan-2-yl)methyl)phenyl)pyridin-4-yl)phenyl)-2-methoxypyridin-3-yl)methyl)-2,6-diazaspiro[3.4]octan-7-one (K), or a salt or solvate thereof.

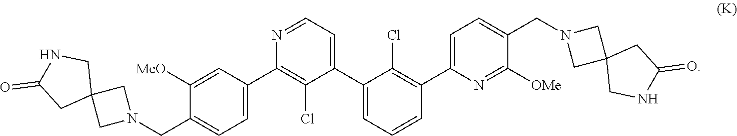 [SMILES: COc1cc(-c2nccc(-c3cccc(-c4ccc(CN5CC6(CNC(=O)C6)C5)c(OC)n4)c3Cl)c2Cl)ccc1CN1CC2(CNC(=O)C2)C1]

### DETAILED DESCRIPTION OF THE INVENTION

This disclosure relates, in certain aspects, to the discovery of scalable synthetic routes that allow for reproducible multi-gram synthesis of certain substituted 1-aryl-1′-heteroaryl and substituted 1,1′-biheteroaryl compounds that are useful to treat, ameliorate, and/or prevent hepatitis B virus (HBV) and/or hepatitis D virus (HDV) infection and related conditions in a subject.

The disclosure of PCT International Patent Application No. PCT/IB2022/052782, filed Mar. 25, 2022, U.S. Provisional Application No. 63/167,440, filed Mar. 29, 2021, and U.S. Provisional Application No. 63/291,666, filed Dec. 20, 2021 are incorporated herein by reference in their entireties.

### Definitions

As used herein, each of the following terms has the meaning associated with it in this section. Unless defined otherwise, all technical and scientific terms used herein generally have the same meaning as commonly understood by one of ordinary skill in the art to which this disclosure belongs. Generally, the nomenclature used herein and the laboratory procedures in animal pharmacology, pharmaceutical science, separation science, and organic chemistry are those well-known and commonly employed in the art. It should be understood that the order of steps or order for performing certain actions is immaterial, so long as the present teachings remain operable. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the application, where an element or component is said to be included in and/or selected from a list of recited elements or components, it should be understood that the element or component can be any one of the recited elements or components and can be selected from a group consisting of two or more of the recited elements or components.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” As used herein, the term “about” will be understood by persons of ordinary skill in the art and will vary to some extent on the context in which it is used. As used herein, “about” when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

As used herein, the term “alkenyl,” employed alone or in combination with other terms, means, unless otherwise stated, a stable monounsaturated or diunsaturated straight chain or branched chain hydrocarbon group having the stated number of carbon atoms. Examples include vinyl, propenyl (or allyl), crotyl, isopentenyl, butadienyl, 1,3-pentadienyl, 1,4-pentadienyl, and the higher homologs and isomers. A functional group representing an alkene is exemplified by —CH₂—CH═CH₂.

As used herein, the term “alkoxy” employed alone or in combination with other terms means, unless otherwise stated, an alkyl group having the designated number of carbon atoms, as defined elsewhere herein, connected to the rest of the molecule via an oxygen atom, such as, for example, methoxy, ethoxy, 1-propoxy, 2-propoxy (or isopropoxy) and the higher homologs and isomers. A specific example is (C₁-C₃)alkoxy, such as, but not limited to, ethoxy and methoxy.

As used herein, the term “alkyl” by itself or as part of another substituent means, unless otherwise stated, a straight or branched chain hydrocarbon having the number of carbon atoms designated (i.e., C₁-C₁₀ means one to ten carbon atoms) and includes straight, branched chain, or cyclic substituent groups. Examples include methyl, ethyl, propyl, isopropyl, butyl, isobutyl, tert-butyl, pentyl, neopentyl, hexyl, and cyclopropylmethyl. A specific embodiment is (C₁-C₆)alkyl, such as, but not limited to, ethyl, methyl, isopropyl, isobutyl, n-pentyl, n-hexyl, and cyclopropylmethyl.

As used herein, the term “alkynyl” employed alone or in combination with other terms means, unless otherwise stated, a stable straight chain or branched chain hydrocarbon group with a triple carbon-carbon bond, having the stated number of carbon atoms. Non-limiting examples include ethynyl and propynyl, and the higher homologs and isomers. The term “propargylic” refers to a group exemplified by —CH₂—C≡CH. The term “homopropargylic” refers to a group exemplified by —CH₂CH₂—C≡CH.

As used herein, the term “aromatic” refers to a carbocycle or heterocycle with one or more polyunsaturated rings and having aromatic character, i.e., having (4n+2) delocalized R (pi) electrons, where ‘n’ is an integer.

As used herein, the term “aryl” employed alone or in combination with other terms means, unless otherwise stated, a carbocyclic aromatic system containing one or more rings (typically one, two or three rings) wherein such rings may be attached together in a pendent manner, such as a biphenyl, or may be fused, such as naphthalene. Examples include phenyl, anthracyl and naphthyl. Aryl groups also include, for example, phenyl or naphthyl rings fused with one or more saturated or partially saturated carbon rings (e.g., bicyclo[4.2.0]octa-1,3,5-trienyl, or indanyl), which can be substituted at one or more carbon atoms of the aromatic and/or saturated or partially saturated rings.

As used herein, the term “aryl-(C₁-C₆)alkyl” refers to a functional group wherein a one-to-six carbon alkylene chain is attached to an aryl group, e.g., —CH₂CH₂-phenyl or —CH₂-phenyl (or benzyl). Specific examples are aryl-CH₂— and aryl-CH(CH₃)—. The term “substituted aryl-(C₁-C₆)alkyl” refers to an aryl-(C₁-C₆)alkyl functional group in which the aryl group is substituted. A specific example is substituted aryl(CH₂)—. Similarly, the term “heteroaryl-(C₁-C₆)alkyl” refers to a functional group wherein a one-to-three carbon alkylene chain is attached to a heteroaryl group, e.g., —CH₂CH₂-pyridyl. A specific example is heteroaryl-(CH₂)—. The term “substituted heteroaryl-(C₁-C₆)alkyl” refers to a heteroaryl-(C₁-C₆)alkyl functional group in which the heteroaryl group is substituted. A specific example is substituted heteroaryl-(CH₂)—.

As used herein, the term “cycloalkyl” by itself or as part of another substituent refers to, unless otherwise stated, a cyclic chain hydrocarbon having the number of carbon atoms designated (i.e., C₃-C₆ refers to a cyclic group comprising a ring group consisting of three to six carbon atoms) and includes straight, branched chain or cyclic substituent groups. Examples of (C₃-C₆)cycloalkyl groups are cyclopropyl, cyclobutyl, cyclopentyl and cyclohexyl. Cycloalkyl rings can be optionally substituted. Non-limiting examples of cycloalkyl groups include: cyclopropyl, 2-methyl-cyclopropyl, cyclopropenyl, cyclobutyl, 2,3-dihydroxycyclobutyl, cyclobutenyl, cyclopentyl, cyclopentenyl, cyclopentadienyl, cyclohexyl, cyclohexenyl, cycloheptyl, cyclooctanyl, decalinyl, 2,5-dimethylcyclopentyl, 3,5-dichlorocyclohexyl, 4-hydroxycyclohexyl, 3,3,5-trimethylcyclohex-1-yl, octahydropentalenyl, octahydro-1H-indenyl, 3a,4,5,6,7,7a-hexahydro-3H-inden-4-yl, decahydroazulenyl; bicyclo[6.2.0]decanyl, decahydronaphthalenyl, and dodecahydro-1H-fluorenyl. The term “cycloalkyl” also includes bicyclic hydrocarbon rings, non-limiting examples of which include, bicyclo[2.1.1]hexanyl, bicyclo[2.2.1]heptanyl, bicyclo[3.1.1]heptanyl, 1,3-dimethyl[2.2.1]heptan-2-yl, bicyclo[2.2.2]octanyl, and bicyclo[3.3.3]undecanyl.

As used herein, a “disease” is a state of health of a subject wherein the subject cannot maintain homeostasis, and wherein if the disease is not ameliorated then the subject's health continues to deteriorate.

As used herein, a “disorder” in a subject is a state of health in which the subject is able to maintain homeostasis, but in which the subject's state of health is less favorable than it would be in the absence of the disorder. Left untreated, a disorder does not necessarily cause a further decrease in the subject's state of health.

As used herein, the term “halide” refers to a halogen atom bearing a negative charge. The halide anions are fluoride (F⁻), chloride (Cl⁻), bromide (Br⁻), and iodide (I⁻).

As used herein, the term “halo” or “halogen” alone or as part of another substituent refers to, unless otherwise stated, a fluorine, chlorine, bromine, or iodine atom.

As used herein, the term “heteroalkenyl” by itself or in combination with another term refers to, unless otherwise stated, a stable straight or branched chain monounsaturated or diunsaturated hydrocarbon group consisting of the stated number of carbon atoms and one or two heteroatoms selected from the group consisting of O, N, and S, and wherein the nitrogen and sulfur atoms may optionally be oxidized and the nitrogen heteroatom may optionally be quaternized. Up to two heteroatoms may be placed consecutively. Examples include —CH═CH—O—CH₃, —CH═CH—CH₂—OH, —CH₂—CH═N—OCH₃, —CH═CH—N(CH₃)—CH₃, and —CH₂—CH═CH—CH₂—SH.

As used herein, the term “heteroalkyl” by itself or in combination with another term refers to, unless otherwise stated, a stable straight or branched chain alkyl group consisting of the stated number of carbon atoms and one or two heteroatoms selected from the group consisting of O, N, and S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Examples include: —OCH₂CH₂CH₃, —CH₂CH₂CH₂OH, —CH₂CH₂NHCH₃, —CH₂SCH₂CH₃, and —CH₂CH₂S(═O)CH₃. Up to two heteroatoms may be consecutive, such as, for example, —CH₂NH—OCH₃, or —CH₂CH₂SSCH₃.

As used herein, the term “heteroaryl” or “heteroaromatic” refers to a heterocycle having aromatic character. A polycyclic heteroaryl may include one or more rings that are partially saturated. Examples include tetrahydroquinoline and 2,3-dihydrobenzofuryl.

As used herein, the term “heterocycle” or “heterocyclyl” or “heterocyclic” by itself or as part of another substituent refers to, unless otherwise stated, an unsubstituted or substituted, stable, mono- or multi-cyclic heterocyclic ring system that comprises carbon atoms and at least one heteroatom selected from the group consisting of N, O, and S, and wherein the nitrogen and sulfur heteroatoms may be optionally oxidized, and the nitrogen atom may be optionally quaternized. The heterocyclic system may be attached, unless otherwise stated, at any heteroatom or carbon atom that affords a stable structure. A heterocycle may be aromatic or non-aromatic in nature. In certain embodiments, the heterocycle is a heteroaryl.

Examples of non-aromatic heterocycles include monocyclic groups such as aziridine, oxirane, thiirane, azetidine, oxetane, thietane, pyrrolidine, pyrroline, imidazoline, pyrazolidine, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, homopiperidine, 1,3-dioxepane, 4,7-dihydro-1,3-dioxepin, and hexamethyleneoxide.

Examples of heteroaryl groups include pyridyl, pyrazinyl, pyrimidinyl (such as, but not limited to, 2- and 4-pyrimidinyl), pyridazinyl, thienyl, furyl, pyrrolyl, imidazolyl, thiazolyl, oxazolyl, pyrazolyl, isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl, and 1,3,4-oxadiazolyl.

Examples of polycyclic heterocycles include indolyl (such as, but not limited to, 2-, 3-, 4-, 5-, 6- and 7-indolyl), indolinyl, quinolyl, tetrahydroquinolyl, isoquinolyl (such as, but not limited to, 1- and 5-isoquinolyl), 1,2,3,4-tetrahydroisoquinolyl, cinnolinyl, quinoxalinyl (such as, but not limited to, 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (such as, but not limited to, 3-, 4-, 5-, 6- and 7-benzofuryl), 2,3-dihydrobenzofuryl, 1,2-benzisoxazolyl, benzothienyl (such as, but not limited to, 3-, 4-, 5-, 6-, and 7-benzothienyl), benzoxazolyl, benzothiazolyl (such as, but not limited to, 2-benzothiazolyl and 5-benzothiazolyl), purinyl, benzimidazolyl, benztriazolyl, thioxanthinyl, carbazolyl, carbolinyl, acridinyl, pyrrolizidinyl, and quinolizidinyl.

The aforementioned listing of heterocyclyl and heteroaryl moieties is intended to be representative and not limiting.

As used herein, the term “pharmaceutical composition” or “composition” refers to a mixture of at least one compound useful within the disclosure with a pharmaceutically acceptable carrier. The pharmaceutical composition facilitates administration of the compound to a subject.

As used herein, the term “pharmaceutically acceptable” refers to a material, such as a carrier or diluent, which does not abrogate the biological activity or properties of the compound useful within the disclosure, and is relatively non-toxic, i.e., the material can be administered to a subject without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the disclosure within or to the subject such that it can perform its intended function. Typically, such constructs are carried or transported from one organ, or portion of the body, to another organ, or portion of the body. Each carrier must be “acceptable” in the sense of being compatible with the other ingredients of the formulation, including the compound useful within the disclosure, and not injurious to the subject. Some examples of materials that can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the disclosure, and are physiologically acceptable to the subject. Supplementary active compounds can also be incorporated into the compositions. The “pharmaceutically acceptable carrier” can further include a pharmaceutically acceptable salt of the compound useful within the disclosure. Other additional ingredients that can be included in the pharmaceutical compositions used in the practice of the disclosure are known in the art and described, for example in Remington's Pharmaceutical Sciences (Genaro, Ed., Mack Publishing Co., 1985, Easton, PA), which is incorporated herein by reference.

As used herein, the language “pharmaceutically acceptable salt” refers to a salt of the administered compound prepared from pharmaceutically acceptable non-toxic acids and/or bases, including inorganic acids, inorganic bases, organic acids, inorganic bases, solvates (including hydrates) and clathrates thereof.

As used herein, a “pharmaceutically effective amount,” “therapeutically effective amount,” or “effective amount” of a compound is that amount of compound that is sufficient to provide a beneficial effect to the subject to which the compound is administered.

The term “prevent,” “preventing,” or “prevention” as used herein means avoiding or delaying the onset of symptoms associated with a disease or condition in a subject that has not developed such symptoms at the time the administering of an agent or compound commences. Disease, condition and disorder are used interchangeably herein.

By the term “specifically bind” or “specifically binds” as used herein is meant that a first molecule preferentially binds to a second molecule (e.g., a particular receptor or enzyme), but does not necessarily bind only to that second molecule.

As used herein, the terms “subject” and “individual” and “patient” can be used interchangeably and can refer to a human or non-human mammal or a bird. Non-human mammals include, for example, livestock and pets, such as ovine, bovine, porcine, canine, feline and murine mammals. In certain embodiments, the subject is human.

As used herein, the term “substituted” refers to that an atom or group of atoms has replaced hydrogen as the substituent attached to another group.

As used herein, the term “substituted alkyl,” “substituted cycloalkyl,” “substituted alkenyl,” or “substituted alkynyl” refers to alkyl, cycloalkyl, alkenyl, or alkynyl, as defined elsewhere herein, substituted by one, two or three substituents independently selected from the group consisting of halogen, —OH, alkoxy, tetrahydro-2-H-pyranyl, —NH₂, —NH(C₁-C₆ alkyl), —N(C₁-C₆ alkyl)₂, 1-methyl-imidazol-2-yl, pyridin-2-yl, pyridin-3-yl, pyridin-4-yl, —C(═O)OH, —C(═O)O(C₁-C₆)alkyl, trifluoromethyl, —C≡N, —C(═O)NH₂, —C(═O)NH(C₁-C₆)alkyl, —C(═O)N((C₁-C₆)alkyl)₂, —SO₂NH₂, —SO₂NH(C₁-C₆ alkyl), —SO₂N(C₁-C₆ alkyl)₂, —C(═NH)NH₂, and —NO₂, in certain embodiments containing one or two substituents independently selected from halogen, —OH, alkoxy, —NH₂, trifluoromethyl, —N(CH₃)₂, and —C(═O)OH, in certain embodiments independently selected from halogen, alkoxy and —OH. Examples of substituted alkyls include, but are not limited to, 2,2-difluoropropyl, 2-carboxycyclopentyl and 3-chloropropyl.

For aryl, aryl-(C₁-C₃)alkyl and heterocyclyl groups, the term “substituted” as applied to the rings of these groups refers to any level of substitution, namely mono-, di-, tri-, tetra-, or penta-substitution, where such substitution is permitted. The substituents are independently selected, and substitution can be at any chemically accessible position. In certain embodiments, the substituents vary in number between one and four. In other embodiments, the substituents vary in number between one and three. In yet another embodiments, the substituents vary in number between one and two. In yet other embodiments, the substituents are independently selected from the group consisting of C₁-C₆ alkyl, —OH, C₁-C₆ alkoxy, halogen, amino, acetamido and nitro. As used herein, where a substituent is an alkyl or alkoxy group, the carbon chain can be branched, straight or cyclic.

Unless otherwise noted, when two substituents are taken together to form a ring having a specified number of ring atoms (e.g., R² and R³ taken together with the nitrogen to which they are attached to form a ring having from 3 to 7 ring members), the ring can have carbon atoms and optionally one or more (e.g., 1 to 3) additional heteroatoms independently selected from nitrogen, oxygen, or sulfur. The ring can be saturated or partially saturated, and can be optionally substituted.

Whenever a term or either of their prefix roots appear in a name of a substituent the name is to be interpreted as including those limitations provided herein. For example, whenever the term “alkyl” or “aryl” or either of their prefix roots appear in a name of a substituent (e.g., arylalkyl, alkylamino) the name is to be interpreted as including those limitations given elsewhere herein for “alkyl” and “aryl” respectively.

In certain embodiments, substituents of compounds are disclosed in groups or in ranges. It is specifically intended that the description include each and every individual subcombination of the members of such groups and ranges. For example, the term “C₁₋₆ alkyl” is specifically intended to individually disclose C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl.

The terms “treat,” “treating” and “treatment,” as used herein, means reducing the frequency or severity with which symptoms of a disease or condition are experienced by a subject by virtue of administering an agent or compound to the subject.

Certain abbreviations used herein follow: cccDNA, covalently closed circular DNA; DMSO, dimethylsulfoxide; HBsAg, HBV surface antigen; HBV, hepatitis B virus; HDV, hepatitis D virus; HPLC, high pressure liquid chromatography; LCMS, liquid chromatography mass spectrometry; NMR, Nuclear Magnetic Resonance; pg RNA, pregenomic RNA; RT, retention time; sAg, surface antigen; TLC, thin layer chromatography.

Ranges: throughout this disclosure, various aspects of the present disclosure can be presented in a range format. It should be understood that the description in range format is merely for convenience and brevity and should not be construed as an inflexible limitation on the scope of the present disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise. This applies regardless of the breadth of the range.

### Synthesis

The present disclosure further provides methods of preparing certain compounds of the present disclosure. Compounds of the present teachings can be prepared in accordance with the procedures outlined herein, from commercially available starting materials, compounds known in the literature, or readily prepared intermediates, by employing standard synthetic methods and procedures known to those skilled in the art. Standard synthetic methods and procedures for the preparation of organic molecules and functional group transformations and manipulations can be readily obtained from the relevant scientific literature or from standard textbooks in the field.

It is appreciated that where typical or preferred process conditions (i.e., reaction temperatures, times, mole ratios of reactants, solvents, pressures, and so forth) are given, other process conditions can also be used unless otherwise stated. Optimum reaction conditions can vary with the particular reactants or solvent used, but such conditions can be determined by one skilled in the art by routine optimization procedures. Those skilled in the art of organic synthesis will recognize that the nature and order of the synthetic steps presented can be varied for the purpose of optimizing the formation of the compounds described herein.

The processes described herein can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatography such as high-performance liquid chromatography (HPLC), gas chromatography (GC), gel-permeation chromatography (GPC), or thin layer chromatography (TLC).

Preparation of the compounds can involve protection and deprotection of various chemical groups. The need for protection and deprotection and the selection of appropriate protecting groups can be readily determined by one skilled in the art. The chemistry of protecting groups can be found, for example, in Greene, et al., Protective Groups in Organic Synthesis, 2d. Ed. (Wiley & Sons, 1991), the entire disclosure of which is incorporated by reference herein for all purposes.

The reactions or the processes described herein can be carried out in suitable solvents that can be readily selected by one skilled in the art of organic synthesis. Suitable solvents typically are substantially nonreactive with the reactants, intermediates, and/or products at the temperatures at which the reactions are carried out, i.e., temperatures that can range from the solvent's freezing temperature to the solvent's boiling temperature. A given reaction can be carried out in one solvent or a mixture of more than one solvent. Depending on the particular reaction step, suitable solvents for a particular reaction step can be selected.

The disclosure includes methods of preparing 2-((6-(2-chloro-3-(3-chloro-2-(3-methoxy-4-((7-oxo-2,6-diazaspiro[3.4]octan-2-yl)methyl)phenyl)pyridin-4-yl)phenyl)-2-methoxypyridin-3-yl)methyl)-2,6-diazaspiro[3.4]octan-7-one, also known as compound (K), or a salt, solvate, prodrug, isotopically labeled derivative, and/or tautomer thereof, and any mixtures thereof:

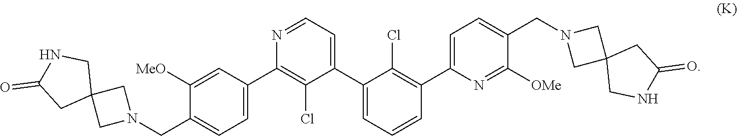 [SMILES: COc1cc(-c2nccc(-c3cccc(-c4ccc(CN5CC6(CNC(=O)C6)C5)c(OC)n4)c3Cl)c2Cl)ccc1CN1CC2(CNC(=O)C2)C1]

In certain embodiments, the compound of formula (K), or a salt, solvate, prodrug, isotopically labeled derivative, and/or tautomer thereof, can be prepared according to the non-limiting synthetic schemes outlined in Schemes 1-3, wherein each occurrence of Z¹ is independently selected from the group consisting of Br and I, wherein Z² is selected from the group consisting of Cl, Br, or I, and wherein R is selected from the group consisting of H, C₁-C₆ alkyl, and C₃-C₈ cycloalkyl, wherein any two R groups may combine with the atoms to which they are bound to form a C₃-C₄ heterocycloalkyl.

Commercially available 1-Z¹-2-chloro-3-Z¹-benzene (A) can be converted to boronic ester or boronic acid (B), for example, by reaction of (A) and a suitable organomagnesium halide, including but not limited to a iPrMg·LiCl (i.e., Turbo-Grignard), at a suitable temperature, including but not limited to about −25° C. to about −10° C., in the presence of a suitable solvent, including but not limited to 2-methyltetrahydrofuran (MeTHF), to provide an organomagnesium intermediate which is reacted with a borate, including but not limited to 2-isopropoxy-4,4,5,5-tetramethyl-1,3,2-dioxaborolane, at a suitable temperature, including but not limited to about −20° C. to about −10° C. In certain embodiments, borate (B) may be converted to compound (D) without purification, for example, using a solution compound (B) generated in situ, as described herein. In other embodiments, compound (B) may be isolated and/or purified.

Compound (D) can be prepared by reaction of compound (B) and heteroaryl halide (C) in the presence of a suitable palladium catalyst, including but not limited to Pd(PPh₃)₄, a suitable base, including but not limited to K₂CO₃, and a suitable solvent, including but not limited to a mixture of MeTHF and water, under suitable reaction conditions, including but not limited to having a temperature of about 50° C. to about 60° C. Thus, in certain embodiments, compound (D) may be prepared from compound (A) without purification of an intermediate.

Compound (F) can be prepared by reaction of aromatic halide (D) and boronic ester or boronic acid (E), in the presence of a suitable palladium catalyst, including but not limited to Pd(amphos)Cl₂ (i.e., bis(di-tert-butyl(4-dimethylaminophenyl)phosphine)dichloropalladium(II)), a suitable base, including but not limited to K₂HPO₄, and a suitable solvent, including but not limited to a mixture of MeTHF, water, and dimethylacetamide (DMAc), under suitable conditions, including but not limited to having a temperature of about 65° C. to about 70° C.

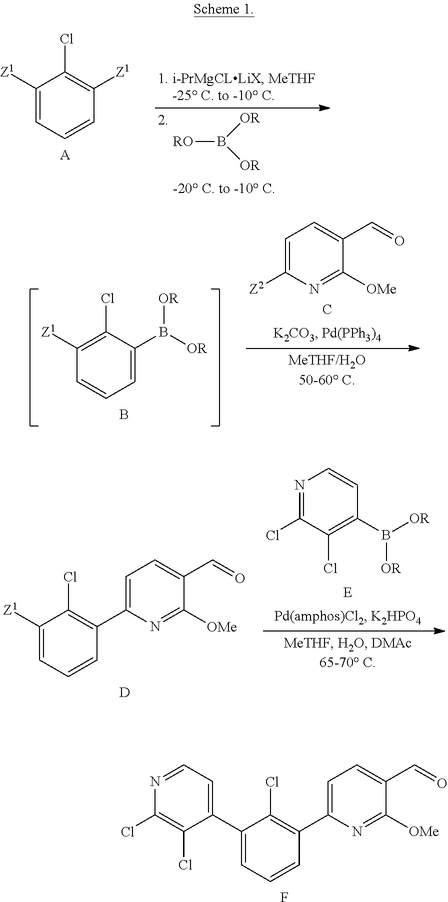

In certain embodiments, compound (F) may be prepared by an alternative sequence, as provided in Scheme 2.

Compound (D′) can be prepared by the reaction of compound (D) and a suitable borylating reagent, including but not limited to bis(pinacolato)diboron (i.e., B₂Pin₂), in the presence of a suitable palladium catalyst, including but not limited to Pd(dppf)Cl·CH₂Cl₂, a suitable base, including but not limited to KOAc, and a suitable solvent, including but not limited to a mixture of dimethylformamide (DMF) and toluene, under suitable reaction conditions, including but not limited to having a temperature of about 90° C. to about 100° C.

Compound (F) can be prepared by the reaction of (D′) and (E′) in the presence of a suitable palladium catalyst, including but not limited to Pd(dppf)Cl·CH₂Cl₂, a suitable base, including but not limited to K₂CO₃, and a suitable solvent, including but not limited to a mixture comprising MeTHF and water, under suitable reaction conditions, including but not limited to having a temperature of about 55° C.

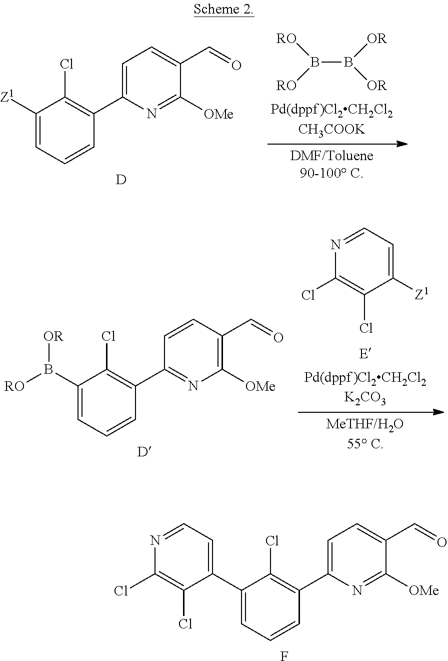

Compound (H) can be prepared by the reaction of compound (G) and suitable borylating reagent, including but not limited to bis(pinacolato)diboron (i.e., B₂Pin₂), in the presence of a suitable palladium catalyst, including but not limited to Pd(dppf)Cl·CH₂Cl₂, a suitable base, including but not limited to KOAc, and a suitable solvent, including but not limited to a mixture of dimethylformamide (DMF) and toluene, under suitable reaction conditions, including but not limited to having a temperature of about 80° C. to about 90° C.

Compound (I) can be prepared by the reaction of compound (H) and compound (F) in the presence of a suitable palladium catalyst, including but not limited to Pd(PPh₃)₄, a suitable base, including but not limited to K₂CO₃, and a suitable solvent, including but not limited to a mixture comprising MeTHF, DMF, and water, under suitable reaction conditions, including but not limited to having a temperature of about 65° C. to about 75° C.

Compound (K) can be prepared by the reaction of compound (I) and compound (J) in the presence of a suitable reducing agent, including but not limited to NaBH(OAc)₃, a suitable base, including but not limited to NaOMe and i-Pr₂NEt, and a suitable solvent, including but not limited to a mixture of DCM and methanol MeOH and/or a mixture of MeOH and MeTHF.

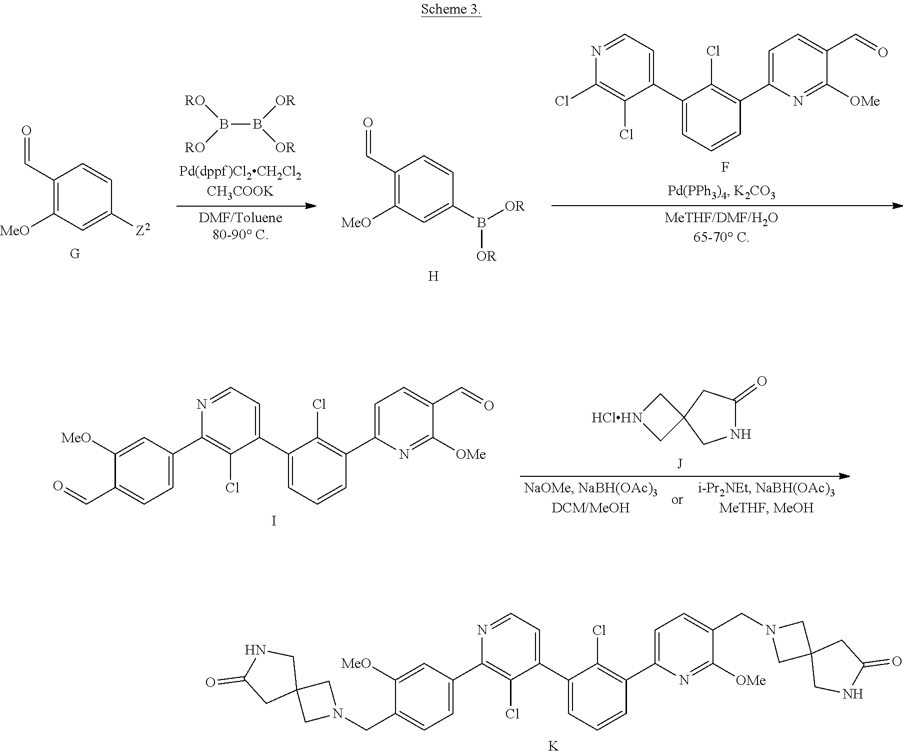

It is understood that the reactions or processes described herein for preparing 2-((6-(2-chloro-3-(3-chloro-2-(3-methoxy-4-((7-oxo-2,6-diazaspiro[3.4]octan-2-yl)methyl)phenyl)pyridin-4-yl)phenyl)-2-methoxypyridin-3-yl)methyl)-2,6-diazaspiro[3.4]octan-7-one (K) are not limited to the embodiments described in Schemes 1-3.

In one aspect, the present disclosure provides a method of preparing 2-((6-(2-chloro-3-(3-chloro-2-(3-methoxy-4-((7-oxo-2,6-diazaspiro[3.4]octan-2-yl)methyl)phenyl)pyridin-4-yl)phenyl)-2-methoxypyridin-3-yl)methyl)-2,6-diazaspiro[3.4]octan-7-one (K), or a salt or solvate thereof

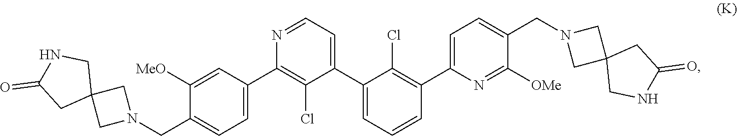 [SMILES: COc1cc(-c2nccc(-c3cccc(-c4ccc(CN5CC6(CNC(=O)C6)C5)c(OC)n4)c3Cl)c2Cl)ccc1CN1CC2(CNC(=O)C2)C1]

the method comprising reacting 6-(2-chloro-3-(3-chloro-2-(4-formyl-3-methoxyphenyl)pyridin-4-yl)phenyl)-2-methoxynicotinaldehyde (I):

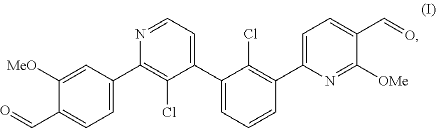 [SMILES: COc1cc(-c2nccc(-c3cccc(-c4ccc(C=O)c(OC)n4)c3Cl)c2Cl)ccc1C=O]

and 2,6-diazaspiro[3.4]octan-7-one (J):

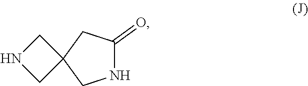

in the presence of a reducing agent and a base, so as to generate a first reaction system comprising (K).

In certain embodiments, the reducing agent comprises NaBH(OAc)₃.

In certain embodiments, the base comprises NaOMe. In certain embodiments, the base comprises i-PrNEt₂.

In certain embodiments, (J) is a salt of 2,6-diazaspiro[3.4]octan-7-one. In certain embodiments, (J) is 2,6-diazaspiro[3.4]octan-7-one hydrochloride. In certain embodiments, (J) is 2,6-diazaspiro[3.4]octan-7-one hydrobromide. In certain embodiments, (J) is 2,6-diazaspiro[3.4]octan-7-one trifluoroacetate. In certain embodiments, (J) is 2,6-diazaspiro[3.4]octan-7-one mesylate. In certain embodiments, (J) is 2,6-diazaspiro[3.4]octan-7-one tosylate.

In certain embodiments, the reaction of (I) and (J) occurs in the presence of a solvent.

In certain embodiments, the solvent is a mixture comprising dichloromethane (DCM) and methanol (MeOH). In certain embodiments, the solvent is a mixture comprising 2-methyltetrahydrofuran (MeTHF) and MeOH. In certain embodiments, the solvent comprises tetrahydrofuran (THF). In certain embodiments, the solvent comprises dimethylformamide (DMF). In certain embodiments, the solvent comprises dimethylacetamide (DMAc). In certain embodiments, the solvent comprises a mixture of any of DCM, MeOH, MeTHF, THF, DMF, and DMAc.

In certain embodiments, purification of (K) comprises:
(a) adding water to the first reaction system comprising (K) to generate a biphasic solution;
(b) separating the biphasic solution to provide a first aqueous phase and a first organic phase;
(c) adding an organic solvent to the first aqueous solution to provide a second biphasic solution;
(d) basifying the second biphasic solution to pH 8-11 to provide a basified biphasic solution; and
(e) separating the basified biphasic solution to provide a second aqueous phase and a second organic phase comprising (K).

In certain embodiments, purification of (K) comprises:
(a) adding water to the first reaction system comprising (K) to generate a biphasic solution;
(b) separating the biphasic solution to provide a first aqueous phase and a first organic phase;
(c) adding an organic solvent to the first aqueous solution to provide a second biphasic solution;
(d) adjusting the second biphasic solution to pH 8-11 to provide a basified biphasic solution;
(e) separating the basified biphasic solution to provide a second aqueous phase and a second organic phase;
(f) combining the first organic phase and the second organic phase to provide a third organic phase;
(g) adding water to the third organic phase to provide a third biphasic solution; and
(h) separating the third biphasic solution to provide a third aqueous phase and a final organic phase comprising (K).

In certain embodiments, the biphasic solution is agitated. In certain embodiments, the biphasic solution is agitated for a period of about 20 min.

In certain embodiments, the organic solvent comprises dichloromethane.

In certain embodiments, the basifying comprises adding a base selected from the group consisting of NaOH, KOH, LiOH, NaHCO₃, K₂CO₃, CaCO₃, Na₂CO₃, and K₃PO₄. In certain embodiments, the NaOH is 3 N NaOH.

In certain embodiments, purification of (K) comprises:
(a) providing crude (K) in a solvent comprising 2-propanol to afford a dilute crude solution of (K),
   wherein the dilute crude solution of (K) has a concentration of about 110 g/L to about 140 g/L;
(b) at least partially evaporating the dilute crude solution of (K) to provide a concentrated crude solution of (K),
   wherein the concentrated crude solution of (K) has a concentration of about 220 g/L to about 280 g/L; and
(c) cooling the concentrated crude solution of (K) to a temperature of about 20° C. provide a purified (K) slurry; and
(d) filtering the purified slurry of (K) to provide (K).

In certain embodiments, purification of (K) comprises:
(a) providing crude (K) in a solvent comprising 2-propanol to afford a dilute crude solution of (K),
   wherein the dilute crude solution of (K) has a concentration of about 110 g/L to about 140 g/L;
(b) at least partially evaporating the dilute crude solution of (K) to provide a concentrated crude solution of (K),
   wherein the concentrated crude solution of (K) has a concentration of about 220 g/L to about 280 g/L; and
   wherein the at least partial evaporation optionally comprises heating the dilute crude solution of (K) to a temperature of about 40° C. to 50° C., optionally for a period of about 2 h;
(c) adding a seed crystal of (K) in an amount of about 0.1% to 1.0% w/w of crude (K) to provide a crude (K) slurry;
(d) heating the crude (K) slurry at a temperature of about 50° C. to provide a heated crude (K) slurry;
(e) cooling the heated crude (K) slurry to a temperature of about 20° C. provide a cooled crude (K) slurry,
   wherein the cooling of the heated crude (K) slurry occurs for a period of about 10 h;
(f) stirring the cooled crude (K) slurry at a temperature of about 20° C. for a period of about 4 h to provide a purified slurry of (K)
   wherein the stirring of the cooled crude (K) occurs for a period of about 4 h;
(g) filtering the purified slurry of (K) to provide (K).

In certain embodiments, purification of (K) comprises:
(a) providing crude (K) in a solvent comprising MeOH to afford a crude solution of (K),
   wherein the crude solution of (K) has a concentration of about 200 g/L to about 300 g/L;
(b) heating the crude solution of (K) to a temperature of about 50° C. to provide a hot crude solution of (K);
(c) cooling the hot crude solution of (K) to a temperature of about 20° C. provide a cooled (K) slurry;
(d) adding a solvent comprising methyl tert-butyl ether (MTBE) to the cooled (K) slurry to provide a purified (K) slurry; and
(e) filtering the purified (K) slurry to provide (K).

In certain embodiments, purification of (K) comprises:
(a) adding an aqueous solution of NaCl to the first reaction system comprising (K) to generate a biphasic solution;
(b) separating the biphasic solution to provide a first aqueous phase and a first organic phase;
(c) washing the first aqueous phase with one or more organic solvents one or more times to provide a washed aqueous phase,
   wherein the one or more organic solvents optionally comprise MeTHF and/or MEK;
(d) adding an organic solvent to the washed aqueous phase to provide a second biphasic solution,
   wherein the organic solvent optionally comprises MEK;
(e) basifying the second biphasic solution to pH of about 9.5-10.5 to provide a basified second biphasic solution,
   wherein the second biphasic solution is optionally basified with an aqueous solution,
   wherein the aqueous solution optionally comprises at least one selected from the group consisting of Na₂CO₃, NaOH, KOH, LiOH, K₂CO₃, CaCO₃, Na₂CO₃, and K₃PO₄;
(f) separating the basified second biphasic solution to provide a second aqueous phase and a second organic phase,
   wherein the second aqueous phase is optionally washed with an organic solvent to provide a third organic phase which is combined with the second organic phase,
   wherein the second organic phase is optionally washed with an aqueous solution of NaCl,
   wherein the second organic phase is optionally filtered through celite;
(g) at least partially evaporating the second organic phase and adding MeOH one or more times to obtain a solution of crude (K) having a concentration of about 200 g/L to about 300 g/L;
(h) heating the crude solution of (K) to a temperature of about 50° C. to provide a hot crude solution of (K),
   wherein the hot crude solution of (K) is optionally agitated for a period of about 3 h,
   wherein a seed crystal of (K) is optionally added to the hot crude solution of (K);
(i) cooling the hot crude solution of (K) to a temperature of about 20° C. to provide a cooled crude solution of (K),
   wherein the cooling occurs over a period of about 4 h;
(j) adding methyl tert-butyl ether (MTBE) to the cooled solution of (K) to provide a crude slurry of (K); and
(k) filtering the crude slurry of (K) to provide (K),
   wherein the (K) is optionally washed with a mixture comprising MeOH/MTBE, and
   wherein the (K) is optionally dried in vacuo, optionally for a period of no less than 24 h.

In certain embodiments, (I) is prepared by reacting 2-methoxy-4-(B(OR^{1a})(OR^{1b}))-benzaldehyde (H):

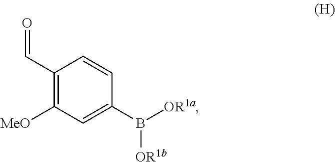

and 6-(2-chloro-3-(2,3-dichloropyridin-4-yl)phenyl)-2-methoxynicotinaldehyde (F):

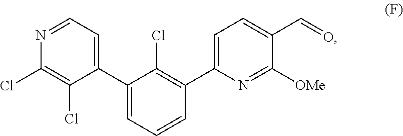 [SMILES: COc1nc(-c2cccc(-c3ccnc(Cl)c3Cl)c2Cl)ccc1C=O]

in the presence of a palladium catalyst and a base;
   wherein each R^{1a} and R^{1b} are each independently selected from the group consisting of C₁-C₆ alkyl and C₃-C₈ cycloalkyl,
      orn R^{1a} and R^{1b} may combine with the atoms to which they are bound to form a C₂-C₃ heterocycloalkyl.

In certain embodiments, the palladium catalyst comprises Pd(PPh₃)₄.

In certain embodiments, the palladium catalyst is present in an amount ranging from about 0.1 mol % to about 5 mol %.

In certain embodiments, the base comprises K₂CO₃.

In certain embodiments, the reaction of (H) and (F) occurs in the presence of a solvent.

In certain embodiments, the solvent comprises at least one of 2-methyltetrahydrofuran (MeTHF), dimethylformamide (DMF), and water.

In certain embodiments, the reaction of (H) and (F) occurs at a temperature of about 65° C. to about 70° C.

In certain embodiments, purification of (I) comprises adding a N-acetyl cysteine to the reaction of (H) and (F). In certain embodiments, the N-acetyl cysteine is added as a solution. In certain embodiments, the N-acetyl cysteine solution is about 1, 2, 3, 4, or 5 wt % N-acetyl cysteine.

In certain embodiments, (H) is prepared by reacting 4-Z²-2-methoxybenzaldehyde (G):

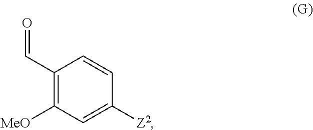

wherein Z² is selected from the group consisting of Cl, Br, and I; and a borylating reagent in the presence of a palladium catalyst and a base.

In certain embodiments, (G) is 4-bromo-2-methoxybenzaldehyde. In certain embodiments, (G) is 4-chloro-2-methoxybenzaldehyde. In certain embodiments, (G) is 4-iodo-2-methoxybenzaldeyde.

In certain embodiments, the borylating reagent is bis(pinacolato)diboron.

In certain embodiments, the palladium catalyst comprises Pd(dppf)Cl₂.

In certain embodiments, the palladium catalyst is present in an amount ranging from about 0.1 mol % to about 5.0 mol %.

In certain embodiments, the base comprises KOAc.

In certain embodiments, the reaction of (G) and the borylating reagent occurs in the presence of a solvent.

In certain embodiments, the solvent comprises dimethylformamide (DMF) and/or toluene.

In certain embodiments, the reaction of (G) and the borylating reagent occurs at a temperature of about 80° C. to about 90° C.

In certain embodiments, purification of (H) comprises at least one of:
(a) adding N-acetyl cysteine to the reaction of (G) and the borylating reagent; and
(b) adding activated carbon to the reaction of (G) and the borylating reagent.

In certain embodiments, the N-acetyl cysteine is added as a solution. In certain embodiments, the N-acetyl cysteine solution is about 1, 2, 3, 4, or 5 wt % N-acetyl cysteine.

In certain embodiments, purification of (H) comprises:
(a) providing crude (H) in a solvent comprising 2-propanol to afford a dilute crude solution of (H),
   wherein the dilute crude solution of (H) has a concentration of about 390 g/L to about 430 g/L;
(b) at least partially evaporating the dilute crude solution of (H) to provide a concentrated crude solution of (H), wherein the at least partial evaporation optionally comprises heating the dilute crude solution of (H) to a temperature of about 50° C., and further optionally maintaining the temperature for a period of about 1.5 h;
(c) cooling the concentrated crude solution of (H) to a temperature of about 6° C., provide a purified (H) slurry, wherein the cooling optionally occurs over a period of about 3 h; and
(d) filtering the purified (H) slurry to provide (H).

In certain embodiments, (F) is prepared by reacting 6-(3-Z¹-2-chlorophenyl)-2-methoxynicotinaldehyde (D):

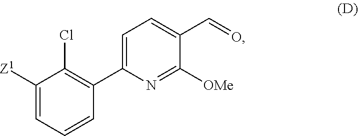

wherein Z¹ is selected from the group consisting of Br and I; and 2,3-dichloro-4-(B(OR^{2a})(OR^{2b}))-pyridine (E):

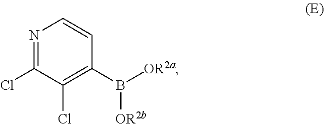

in the presence of a palladium catalyst and a base;
wherein each R^{2a} and R^{2b} are each independently selected from the group consisting of C₁-C₆ alkyl and C₃-C₈ cycloalkyl,
or R^{2a} and R^{2b} may combine with the atoms to which they are bound to form a C₂-C₃ heterocycloalkyl.

In certain embodiments, (D) is 6-(3-bromo-2-chlorophenyl)-2-methoxynicotinaldehyde. In certain embodiments, (D) is 6-(3-iodo-2-chlorophenyl)-2-methoxynicotinaldehyde.

In certain embodiments, R^{2a} is H. In certain embodiments, R^{2b} is H.

In certain embodiments, the palladium catalyst comprises Pd(amphos)Cl₂.

In certain embodiments, the palladium catalyst is present in an amount ranging from about 0.1 mol % to about 5 mol %.

In certain embodiments, the base comprises K₂HPO₄.

In certain embodiments, the reaction of (D) and (E) comprises addition of (E) to a reaction vessel comprising (D), wherein (E) is optionally added to the vessel comprising (D) over a period of about 3.5 h.

In certain embodiments, the reaction of (D) and (E) occurs in the presence of a solvent.

In certain embodiments, the solvent comprises at least one of 2-methyltetrahydrofuran (MeTHF), dimethylacetamide (DMAc), and/or water.

In certain embodiments, the reaction of (D) and (E) occurs at a temperature of about 65° C. to about 70° C.

In certain embodiments, purification of (F) comprises adding a N-acetyl cysteine to the reaction of (D) and (E). In certain embodiments, the N-acetyl cysteine is added as a solution. In certain embodiments, the N-acetyl cysteine solution is about 1, 2, 3, 4, or 5 wt % N-acetyl cysteine.

In certain embodiments, purification of (F) comprises:
(a) providing crude (F) in a solvent comprising 2-propanol to afford a dilute crude solution of (F),
   wherein the dilute crude solution of (F) has a concentration of about 100 g/L to about 150 g/L;
(b) at least partially evaporating the dilute crude solution of (F) to provide a concentrated crude solution of (F), wherein the at least partial evaporation optionally comprises heating the dilute crude solution of (F) to a temperature of about 40° C. to about 55° C., and further optionally maintaining the temperature for a period of about 1 h;
(c) cooling the concentrated crude solution of (F) to a temperature of about 20 to about 25° C., to provide a crude (F) slurry, wherein the cooling optionally occurs over a period of about 3 h; and
(d) filtering the crude (F) slurry to provide (F), wherein the filtering optionally further comprises washing with 2-propanol.

In certain embodiments, purification of (F) further comprises:
(e) providing a solution of (F), as obtained by performing steps (a)-(d) described elsewhere herein, in a solvent comprising 2-propanol to provide a dilute first crop solution,
   wherein the dilute first crop solution of (F) has a concentration of about 100 g/L to about 150 g/L, and
   wherein the dilute first crop solution is obtain by dissolving (F) obtained by performing steps (a)-(d) described elsewhere herein (i.e., first crop (F)) in 2-methyltetrahydrofuran and solvent swapping with 2-propanol;
(f) at least partially evaporating the dilute first crop solution of (F) to provide a concentrated first crop solution of (F), wherein the at least partial evaporation optionally comprises heating the dilute first crop solution of (F) to a temperature of about 40° C. to about 55° C., wherein the heating is optionally maintained for a period of about 1 h;
(g) cooling the concentrated first crop solution of (F) to obtain a first crop (F) slurry, wherein the cooling optionally comprises a temperature reduction to a temperature of about 20° C. to about 25° C., wherein the cooling optionally occurs of a period of about 1 h; and
(h) filtering the first crop (F) slurry to provide a second crop (F) (i.e., purified (F) or (F)), wherein the filtering optionally further comprises washing with a solvent comprising 2-propanol.

In certain embodiments, (F) is prepared by reacting 6-(2-chloro-3-(B(OR^{3a})(OR^{3b}))phenyl)-2-methoxynicotinaldehyde (D′):

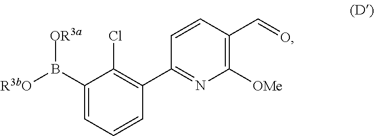

and 2,3-dichloro-4-Z¹-pyridine (E′):

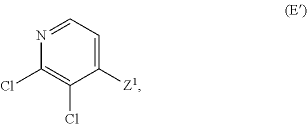

wherein Z¹ is selected from the group consisting of Br and I; in the presence of a palladium catalyst and a base;
wherein each R^{3a} and R^{3b} are each independently selected from the group consisting of C₁-C₆ alkyl and C₃-C₈ cycloalkyl,
or R^{3a} and R^{3b} may combine with the atoms to which they are bound to form a C₂-C₃ heterocycloalkyl.

In certain embodiments, (E′) is 2,3-dichloro-4-iodopyridine. In certain embodiments, (E′) is 2,3-dichloro-4-bromopyridine.

In certain embodiments, (D′) is 6-(2-chloro-3-(4,4,5,5-tetramethyl-1,3,2-dioxaborolan-2-yl)phenyl)-2-methoxynicotinaldehyde.

In certain embodiments, the palladium catalyst comprises Pd(dppf)Cl₂.

In certain embodiments, the palladium catalyst is present in an amount ranging from about 0.1 mol % to about 5 mol %.

In certain embodiments, the base comprises K₂CO₃.

In certain embodiments, reaction of (D′) and (E′) occurs in the presence of a solvent.

In certain embodiments, the solvent comprises 2-methyltetrahydrofuran (MeTHF) and/or water.

In certain embodiments, the reaction of (D′) and (E′) occurs at a temperature of about 55° C.

In certain embodiments, purification of (F) comprises adding N-acetyl cysteine to the reaction of (D′) and (E′). In certain embodiments, the N-acetyl cysteine is added as a solution. In certain embodiments, the N-acetyl cysteine solution is about 1, 2, 3, 4, or 5 wt % N-acetyl cysteine.

In certain embodiments, purification of (F) comprises:
(a) providing crude (F) in 2-propanol to afford a dilute crude solution of (F),
   wherein the dilute crude solution of (F) has a concentration of about 100 g/L to about 140 g/L;
(b) at least partially evaporating the dilute crude solution of (F) to provide a concentrated crude solution of (F), wherein the at least partial evaporation optionally comprises heating the dilute crude solution of (F) to a temperature of about 50° C. to about 55° C., and further optionally maintaining the temperature for a period of about 1 h;
(c) cooling the concentrated crude solution of (F) to a temperature of about 20° C. to about 25° C., to provide a purified (F) slurry; and
(d) filtering the purified (F) slurry to provide (F).

In certain embodiments, (D′) is prepared by reacting (D):

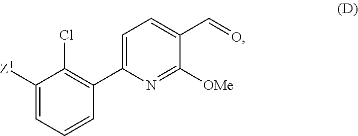

wherein Z¹ is selected from the group consisting of Br and I; and a borylating reagent in the presence of a palladium catalyst and a base.

In certain embodiments, (D) is 6-(3-bromo-2-chlorophenyl)-2-methoxynicotinaldehyde. In certain embodiments, (D) is 6-(3-iodo-2-chlorophenyl)-2-methoxynicotinaldehyde.

In certain embodiments, the borylating reagent comprises bis(pinacolato)diboron.

In certain embodiments, the palladium catalyst comprises Pd(dppf)Cl₂.

In certain embodiments, the palladium catalyst is present in an amount ranging from about 0.1 mol % to about 7 mol %.

In certain embodiments, the base comprises KOAc.

In certain embodiments, the reaction of (D) and the borylating reagent occurs in the presence of a solvent.

In certain embodiments, the solvent comprises at least one of dimethylformamide (DMF) and/or toluene.

In certain embodiments, the reaction of (D) and the borylating reagent occurs at temperature of about 90° C. to about 100° C.

In certain embodiments, purification of (D′) comprises at least one of:
(a) adding a N-acetyl cysteine to the reaction of (D) and the borylating reagent; and
(b) adding activated carbon to the reaction of (D) and the borylating reagent.

In certain embodiments, the N-acetyl cysteine is added as a solution. In certain embodiments, the N-acetyl cysteine solution is about 1, 2, 3, 4, or 5 wt % N-acetyl cysteine.

In certain embodiments, wherein purification of (D′) comprises:
(a) providing crude (D′) in a solvent comprising 2-propanol to afford a crude solution of (D′),
   wherein the crude solution of (D′) has a concentration of about 140 g/L to about 180 g/L;
(c) cooling the crude solution of (D′) to a temperature of about 0° C. to about 5° C., to provide a purified (D′) slurry; and
(d) filtering the purified (D′) slurry to provide (D′).

In certain embodiments, (D) is prepared by reacting 1-(B(OR^{4a})(OR^{4b}))-2-chloro-3-Z¹-benzene (B):

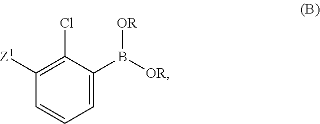

and 6-Z²-2-methoxynicotinaldehyde (C):

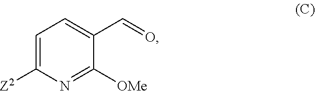

in the presence of a palladium catalyst and a base;
   wherein Z¹ is selected from the group consisting of Br and I;
   wherein Z² is selected from the group consisting of Cl, Br, and I;
   wherein each R^{4a} and R^{4b} are each independently selected from the group consisting of C₁-C₆ alkyl and C₃-C₈ cycloalkyl,
      or R^{4a} and R^{4b} may combine with the atoms to which they are bound to form a C₂-C₃ heterocycloalkyl.

In certain embodiments, (B) is 2-(3-bromo-2-chlorophenyl)-4,4,5,5-tetramethyl-1,3,2-dioxaborolane. In certain embodiments, (B) is 2-(3-iodo-2-chlorophenyl)-4,4,5,5-tetramethyl-1,3,2-dioxaborolane.

In certain embodiments, the palladium catalyst comprises Pd(PPh₃)₄.

In certain embodiments, the palladium catalyst is present in an amount ranging from about 0.1 mol % to about 5 mol %.

In certain embodiments, the base comprises K₂CO₃.

In certain embodiments, the reaction of (B) and (C) occurs in the presence of a solvent.

In certain embodiments, the solvent comprises at least one of 2-methyltetrahydrofuran (MeTHF) and/or water.

In certain embodiments, the reaction of (B) and (C) occurs at a temperature of about 50° C. to about 60° C.

In certain embodiments, wherein purification of (D) comprises adding N-acetyl cysteine to the reaction of (B) and (C). In certain embodiments, the N-acetyl cysteine is added as a solution. In certain embodiments, the N-acetyl cysteine solution is about 1, 2, 3, 4, or 5 wt % N-acetyl cysteine.

In certain embodiments, purification of (D) comprises:
(a) providing crude (D) in solvent comprising 2-propanol to afford a dilute crude solution of (D),
   wherein the dilute crude solution of (D) has a concentration of about 160 g/L to about 220 g/L;
(b) at least partially evaporating the dilute crude solution of (D) to provide a concentrated crude solution of (D), wherein the at least partial evaporation optionally comprises heating the dilute crude solution of (D) to a temperature of about 50° C. to about 55° C., and further optionally maintaining the temperature for a period of about 1 h;
(c) cooling the concentrated crude solution of (D) to a temperature of about 20° C. to about 25° C., to provide a purified (D) slurry; and
(d) filtering the purified (D) slurry to provide (D).

In certain embodiments, (B) is prepared by:
(a) reacting 1-Z¹-2-chloro-3-Z¹-benzene (A):

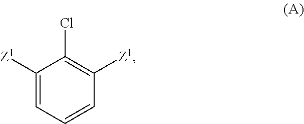

wherein each occurrence of Z¹ is independently selected from the group consisting of Br and I;
and an organomagnesium halide to form an arylmagnesium intermediate; and
(b) reacting the arylmagnesium intermediate with a borate.

In certain embodiments, (A) is 1,3-dibromo-2-chlorobenzene. In certain embodiments, (A) is 1,3-diiodo-2-chlorobenzene. In certain embodiments, (A) is 1-iodo-2-chloro-3-bromobenzene.

In certain embodiments, the borate is:

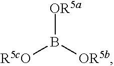

wherein each R^{5a}, R^{5b}, and R^{5c} are each independently selected from the group consisting of C₁-C₆ alkyl and C₃-C₈ cycloalkyl,
   wherein any two selected from the group consisting of R^{5a}, R^{5b}, and R^{5c} may combine with the atoms to which they are bound to form a C₂-C₃ heterocycloalkyl.

In certain embodiments, the borate is 2-isopropoxy-4,4,5,5-tetramethyl-1,3,2-dioxaborolane.

In certain embodiments, the organomagnesium halide comprises i-PrMgCl, wherein the i-PrMgCl is optionally a solution comprising i-PrMgCl complexed with LiCl (i.e., an i-PrMgCl·LiCl solution).

In certain embodiments, the reaction of (A) and the organomagnesium halide occurs in a solvent.

In certain embodiments, the solvent comprises 2-methyltetrahydrofuran (MeTHF).

In certain embodiments, the reaction of (A) and the organomagnesium halide occurs at a temperature of about −25° C. to about −15° C.

In certain embodiments, the reaction of the arylmagnesium intermediate and the borate occurs at a temperature of about −18° C. to about −15° C., wherein the reaction is optionally subsequently warmed to a temperature of about 2° C.

In certain embodiments, (D) is prepared by:
(a) reacting 1-Z¹-2-chloro-3-Z¹-benzene (A):

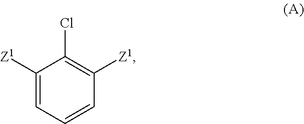

wherein each occurrence of Z¹ is independently selected from the group consisting of Br and I;
and an organomagnesium halide to form an arylmagnesium intermediate;
(b) reacting the arylmagnesium intermediate with a borate to provide a boronic ester intermediate; and
(c) reacting the boronic ester intermediate and 6-Z²-2-methoxynicotinaldehyde (C):

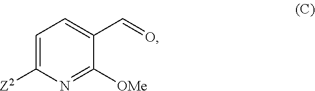

wherein Z² is selected from the group consisting of Cl, Br, and I;
in the presence of a palladium catalyst and a base.

In certain embodiments, the organomagnesium halide comprises i-PrMgCl, wherein the i-PrMgCl is optionally a solution comprising i-PrMgCl complexed with LiCl (i.e., an i-PrMgCl·LiCl solution).

In certain embodiments, the reaction of (A) and the organomagnesium halide occurs in a solvent.

In certain embodiments, the solvent comprises 2-methyltetrahydrofuran (MeTHF).

In certain embodiments, the reaction of (A) and the organomagnesium halide occurs at a temperature of about −25° C. to about −15° C.

In certain embodiments, the reaction of the arylmagnesium intermediate and the borate occurs at a temperature of about −18° C. to about −15° C., wherein the reaction is optionally subsequently warmed to a temperature of about 2° C.

In certain embodiments, (C) is 6-chloro-2-methoxynicotinaldehyde. In certain embodiments, (C) is 6-bromo-2-methoxynicotinaldehyde. In certain embodiments, (C) is 6-iodo-2-methoxynicotinaldehyde.

In certain embodiments, the borate is:

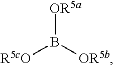

wherein each R^{5a}, R^{5b}, and R^{5c} are each independently selected from the group consisting of C₁-C₆ alkyl and C₃-C₈ cycloalkyl,
   or any two selected from the group consisting of R^{5a}, R^{5b}, and R^{5c} may combine with the atoms to which they are bound to form a C₂-C₃ heterocycloalkyl.

In certain embodiments, the borate is 2-isopropoxy-4,4,5,5-tetramethyl-1,3,2-dioxaborolane.

In certain embodiments, the palladium catalyst comprises Pd(PPh₃)₄.

In certain embodiments, the palladium catalyst is present in an amount ranging from about 0.1 mol % to about 5 mol %.

In certain embodiments, the base comprises K₂CO₃.

In certain embodiments, the reaction of the boronic ester intermediate and (C) occurs in the presence of a solvent.

In certain embodiments, the solvent comprises at least one selected from the group consisting of 2-methyltetrahydrofuran (MeTHF) and water.

In certain embodiments, reaction of the boronic ester intermediate and (C) occurs at a temperature of about 50° C. to about 60° C.

In certain embodiments, purification of (D) comprises adding N-acetyl cysteine to the reaction of (C) and the boronic ester intermediate. In certain embodiments, the N-acetyl cysteine is added as a solution. In certain embodiments, the N-acetyl cysteine solution is about 1, 2, 3, 4, or 5 wt % N-acetyl cysteine.

In certain embodiments, purification of (D) comprises:
(a) providing crude (D) in a solvent comprising 2-propanol to afford a dilute crude solution of (D),
   wherein the dilute crude solution of (D) has a concentration of about 160 g/L to about 220 g/L;
(b) at least partially evaporating the dilute crude solution of (D) to provide a concentrated crude solution of (D), wherein the at least partial evaporation optionally comprises heating the dilute crude solution of (D) to a temperature of about 50° C. to about 55° C., and further optionally maintaining the temperature for a period of about 1 h;
(c) cooling the concentrated crude solution of (D) to a temperature of about 20° C. to about 25° C., to provide a purified (D) slurry; and
(d) filtering the purified (D) slurry to provide (D).

The compounds of the disclosure may possess one or more stereocenters, and each stereocenter may exist independently in either the (R)- or (S)-configuration. In certain embodiments, compounds described herein are present in optically active or racemic forms. The compounds described herein encompass racemic, optically active, regioisomeric and stereoisomeric forms, or combinations thereof that possess the therapeutically useful properties described herein. Preparation of optically active forms is achieved in any suitable manner, including, by way of non-limiting example, by resolution of the racemic form with recrystallization techniques, synthesis from optically active starting materials, chiral synthesis, or chromatographic separation using a chiral stationary phase. A compound illustrated herein by the racemic formula further represents either of the two enantiomers or any mixtures thereof, or in the case where two or more chiral centers are present, all diastereomers or any mixtures thereof.

In certain embodiments, the compounds of the disclosure exist as tautomers. All tautomers are included within the scope of the compounds recited herein.

Compounds described herein also include isotopically labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S. In certain embodiments, substitution with heavier isotopes such as deuterium affords greater chemical stability. Isotopically labeled compounds are prepared by any suitable method or by processes using an appropriate isotopically labeled reagent in place of the non-labeled reagent otherwise employed.

In certain embodiments, the compounds described herein are labeled by other means, including, but not limited to, the use of chromophores or fluorescent moieties, bioluminescent labels, or chemiluminescent labels.

In all of the embodiments provided herein, examples of suitable optional substituents are not intended to limit the scope of the claimed disclosure. The compounds of the disclosure may contain any of the substituents, or combinations of substituents, provided herein.

### Salts

The compounds described herein may form salts with acids or bases, and such salts are included in the present disclosure. The term “salts” embraces addition salts of free acids or bases that are useful within the methods of the disclosure. The term “pharmaceutically acceptable salt” refers to salts that possess toxicity profiles within a range that affords utility in pharmaceutical applications. In certain embodiments, the salts are pharmaceutically acceptable salts. Pharmaceutically unacceptable salts may nonetheless possess properties such as high crystallinity, which have utility in the practice of the present disclosure, such as for example utility in process of synthesis, purification or formulation of compounds useful within the methods of the disclosure.

Suitable pharmaceutically acceptable acid addition salts may be prepared from an inorganic acid or from an organic acid. Examples of inorganic acids include sulfate, hydrogen sulfate, hydrochloric, hydrobromic, hydriodic, nitric, carbonic, sulfuric, and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (or pamoic), methanesulfonic, ethanesulfonic, benzenesulfonic, pantothenic, sulfanilic, 2-hydroxyethanesulfonic, trifluoromethanesulfonic, p-toluenesulfonic, cyclohexylaminosulfonic, stearic, alginic, (3-hydroxybutyric, salicylic, galactaric, galacturonic acid, glycerophosphonic acids and saccharin (e.g., saccharinate, saccharate). Salts may be comprised of a fraction of one, one, or more than one molar equivalent of acid or base with respect to any compound of the disclosure.

Suitable pharmaceutically acceptable base addition salts of compounds of the disclosure include, for example, ammonium salts and metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N-dibenzylethylene-diamine, chloroprocaine, choline, diethanolamine, ethylenediamine, meglumine (or N-methylglucamine) and procaine. All of these salts may be prepared from the corresponding compound by reacting, for example, the appropriate acid or base with the compound.

Those skilled in the art will recognize, or be able to ascertain using no more than routine experimentation, numerous equivalents to the specific procedures, embodiments, claims, and examples described herein. Such equivalents were considered to be within the scope of this disclosure and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that, wherever values and ranges are provided herein, the description in range format is merely for convenience and brevity and should not be construed as an inflexible limitation on the scope of the disclosure. Accordingly, all values and ranges encompassed by these values and ranges are meant to be encompassed within the scope of the present disclosure. Moreover, all values that fall within these ranges, as well as the upper or lower limits of a range of values, are also contemplated by the present application. The description of a range should be considered to have specifically disclosed all the possible sub-ranges as well as individual numerical values within that range and, when appropriate, partial integers of the numerical values within ranges. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The examples provided herein illustrate aspects of the present disclosure. However, they are in no way a limitation of the teachings or disclosure of the present disclosure as set forth herein. The examples herein are provided for the purpose of illustration only, and the disclosure is not limited to these examples, but rather encompasses all variations that are evident as a result of the teachings provided herein.

### EXAMPLES

The disclosure is now described with reference to the following Examples. These Examples are provided for the purpose of illustration only, and the disclosure is not limited to these Examples, but rather encompasses all variations that are evident as a result of the teachings provided herein.

### Step 1—Synthesis of 6-(3-bromo-2-chlorophenyl)-2-methoxynicotinaldehyde

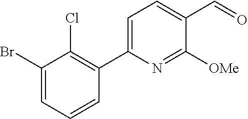 [SMILES: COc1nc(-c2cccc(Br)c2Cl)ccc1C=O]

### (a) Synthesis of 2-(3-bromo-2-chlorophenyl)-4,4,5,5-tetramethyl-1,3,2-dioxaborolane

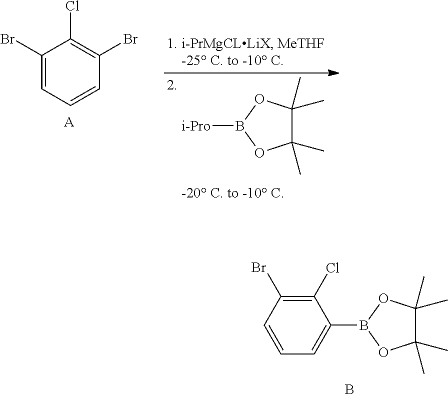

To a 30 L reactor, MeTHF (2.6 L) and i-PrMgCl·LiCl (2.64 L, 3.4 mol, 1.3 M in THF) were charged under nitrogen atmosphere. The resulting solution was allowed to cool to a temperature of about −25° C. to −15° C. (e.g., −24° C.). A solution of compound A (675.5 g, 2.5 mol) in MeTHF (1.06 L) was prepared in a separate 3 L reactor at a temperature of about 8° C. to 25° C. under a nitrogen atmosphere. The solution of compound A was added into a solution of i-PrMgCl·LiCl using addition funnel over a period of about 40 min, while maintaining an internal temperature of about −25° C. to −15° C. The resulting mixture was allowed to agitate at a temperature of about −20° C. to −18° C. for 45 min. Next, 2-isopropoxy-4,4,5,5-tetramethyl-1,3,2-dioxaborolane (593.4 g, 3.2 mol, i.e., i-PrBPin) was added slowly to the reaction mixture, using an addition funnel, over a period of about 40 min, while maintaining an internal temperature of about −18° C. to −15° C. The resulting mixture was allowed to agitate at a temperature of about −17° C. to −15° C. for 30 min. The reaction mixture was then allowed to warm to a temperature of about 2° C. over a period of about 30 min, and allowed to agitate at this temperature for no less than 2 h until completion of the reaction. Reaction completion can be assessed by HPLC, wherein the reaction is deemed complete if no more than 0.5% of compound A remains. The reaction solution was diluted with MeTHF (4.3 L), and water (4.3 L) was charged slowly over a period of 30 min, while maintaining an internal temperature of about 8° C. to 15° C. An aqueous solution of HCl (1.16 L, 3 N) was added to adjust the aqueous layer pH to 6.5. The mixture was allowed to agitate for 30 min, then the layers were separated. The organic layer was washed with 5 wt. % NH₄Cl (3.7 L), 10 wt. % NaCl (3.7 L), dried over MgSO₄ (530 g), and filtered. Filtrate containing compound B was evaporated to about 4.2 L and used in the subsequent reaction without purification.

### (b) Synthesis of 6-(3-bromo-2-chlorophenyl)-2-methoxynicotinaldehyde

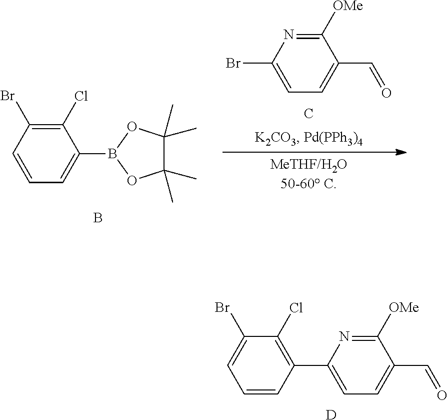

To a 5 L reactor were added K₂CO₃ (1.02 kg, 7.4 mol) and water (1.7 L), and the mixture was agitated to allow solids to dissolve. The atmosphere was evacuated and backfilled with nitrogen gas three times, and the solution was subsequently sparged with nitrogen gas for a period of about 1.5 h. To a separate 30 L reactor, MeTHF (4.8 L) and compound C (530 g, 2.4 mol) were charged and allowed to agitate under nitrogen atmosphere to provide a clear solution, then a solution of compound B, prepared as described elsewhere herein, was added to the solution of compound C. The atmosphere of the vessel comprising the resulting reaction mixture was evacuated and backfilled with nitrogen gas three times, and the reaction mixture was subsequently sparged with nitrogen gas for a period of about 45 min. The K₂CO₃ solution was transferred to the reaction mixture, which was sparged with nitrogen for a period of about 10 min. Next, Pd(PPh₃)₄ (56.7 g, 0.05 mol) was charged and resulting mixture sparged with nitrogen for a period of about 45 min. The reaction mixture was heated to 55° C. and agitated at this temperature for no less than 8 h until completion of the reaction. Reaction completion can be assessed by HPLC, wherein the reaction is deemed complete if no more than 2.0% of compound C remains. Upon completion, MeTHF (5.3 L) was slowly added to the reaction, while maintaining an internal temperature of about 45° C. to 55° C. Next, a 2 wt. % solution of N-acetyl cysteine (4.2 L) was slowly charged to the reaction mixture while maintaining an internal temperature of about 42° C. to 48° C. The contents were allowed to agitate at this temperature for a period of about 1.5 h and layers were allowed to settle. The layers were separated and the organic layer was washed twice with water (4.2 L and 2.1 L) at a temperature of about 42° C. to 45° C. The organic layer was evaporated to a volume of about 2.6 L and diluted with MeTHF to a volume of about 3.7 L. The resulting slurry was subjected to heat cycles, wherein the temperature was increased to about 50° C. and decreased to about 18° C. with stirring, then allowed to stir at a temperature of about 18° C. for about 1.5 h. Solids were filtered, washed with a mixture of MeTHF/n-heptane (1:1, 1.6 L) and dried at a temperature of no more than 50° C. for about 8 h to provide the title compound as an off-white to gray solid (690 g, 86% yield) as off-white to gray solid. H NMR (400 MHz, CDCl₃) δ: 10.41 (s, 1H), 8.18 (d, J=7.8 Hz, 1H), 7.72 (d, J=8.0 Hz, 1H), 7.51 (d, J=7.7 Hz, 1H), 7.30 (d, J=7.8 Hz, 1H), 7.24 (t, J=7.9 Hz, 1H), 4.10 (s, 3H). MS (ESI+, m/z): Calc'd for [C₁₃H₉BrClNO₂+H]⁺: 327.96; Found: 328.1.

### Step 2—Synthesis of 6-(2-chloro-3-(2,3-dichloropyridin-4-yl)phenyl)-2-methoxynicotinaldehyde

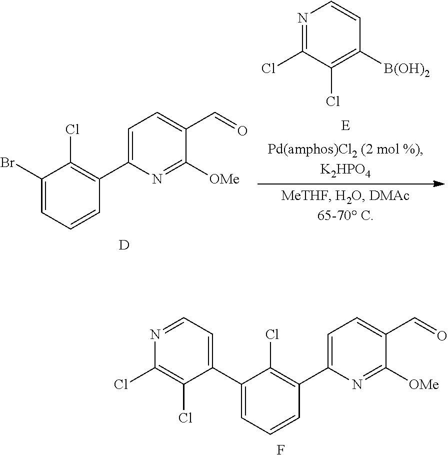

A 10 L reactor was charged with compound D (240 g, 0.73 mol) and MeTHF (4.2 L), and the mixture was agitated to allow solids to dissolve. The atmosphere of the reactor was evacuated and backfilled with nitrogen gas two times, and the solution was subsequently sparged with nitrogen gas for a period of about 0.5 h. To another 2 L reactor, K₂HPO₄ (256 g, 1.5 mol), water (840 mL) were charged and allowed to agitate and dissolve. The atmosphere of the reactor was evacuated and backfilled with nitrogen gas two times, and the solution was subsequently sparged with nitrogen gas for a period of about 1 h. The K₂HPO₄ solution was then transferred to the 10 L reactor under nitrogen atmosphere. The reaction mixture was charged with Pd(amnphos)Cl₂ (10.4 g, 14.7 mmol), and subsequently sparged with nitrogen gas for a period of about 0.5 h. The reaction mixture is heated to a temperature of about 65° C. to 70° C. A separate reactor was charged with compound E (155.2 g, 0.81 mol), 2-MeTHF (600 mL), and DMAc (600 mL), and the mixture was agitated to allow solids to dissolve. The atmosphere of the reactor containing the compound E solution was evacuated and backfilled to nitrogen two times, and the solution was subsequently sparged with nitrogen gas for a period of about 0.5 h. The degassed solution of compound E was slowly transferred into 10 L reaction mixture over a period of about 3.5 h while maintaining an internal temperature of about 65° C. to 70° C. The mixture was agitated at this temperature for a period of about 1 h. Additional compound E (7.0 g, 36.5 mmol) was added to the reaction mixture and heating was continued for no less than 1 h until completion of the reaction. Reaction completion can be assessed by HPLC, wherein the reaction is deemed complete if no more than 2.5% of compound D remains. In certain embodiments, additional compound E may be added to drive the reaction to completion. Next, 2-MeTHF (2.4 L) was charged to the reaction mixture and then the contents were adjusted to a temperature of about 25° C. to 30° C. The reaction mixture was charged with 4 wt. % N-acetyl cysteine solution (2.4 L), allowed to agitate at a temperature of about 20° C. to 30° C. for a period of about 20 min. The layers were separated, and the organic layer was sequentially washed with 10 wt. % NaCl solution (2.4 L), 5 wt. % Na₂CO₃ solution (2.4 L), and water (2.4 L), then dried over Na₂SO₄ and filtered. Filtrate was evaporated at a temperature of about 40° C. to 55° C. and solvent swapped with 2-propanol (2×2.4 L) to provide a solution having a final volume of 1.8 L. The solution was allowed to agitate at a temperature of about 40° C. to 55° C. for 1 h and then allowed to cool to a temperature of about 20° C. to 25° C. and agitated for another 1 h. Solids were filtered, washed with 2-propanol (720 mL) and dried at a temperature of no more than 50° C. to obtain the crude product (241 g).

The crude product was redissolved in MeTHF (4.5 L), evaporated at a temperature of about 40° C. to 55° C., solvent swapped with 2-propanol (2×2.4 L) to a solution with a final volume of 1.8 L. The solution was allowed to agitate at a temperature of about 40° C. to 55° C. for 1 h and then allowed to cool to a temperature of about 20° C. to 25° C. and agitated for another 1 h. The resultant solids were filtered, washed with 2-propanol (720 mL), and dried at a temperature of no more than 50° C. to provide the title compound as an off white solid (213 g, 74% yield). ¹H NMR (400 MHz, DMSO-d₆): δ 10.30 (s, 1H), 8.52 (d, J=4.8 Hz, 1H), 8.23 (d, J=7.6 Hz, 1H), 7.81 (d, J=7.6 Hz, 1H), 7.65-7.55 (m, 3H), 7.48 (d, J=7.6 Hz, 1H), 4.06 (s, 3H). MS (ESI+, m/z): Calc'd for [C₁₈H₁₂C₁₃N₂O₂+]⁺: 393.0; Found: 393.0.

### Step 2′—Synthesis of 6-(2-chloro-3-(4,4,5,5-tetramethyl-1,3,2-dioxaborolan-2-yl)phenyl)-2-methoxynicotinaldehyde

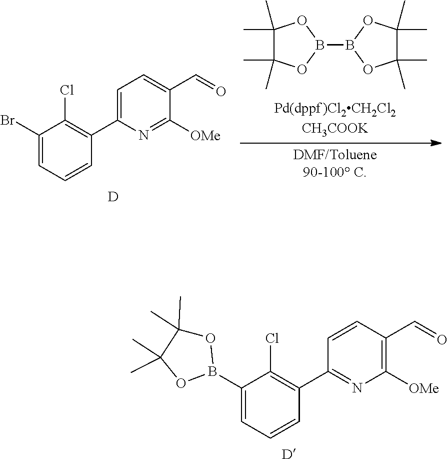

To a 500 mL reactor was added compound D (17.0 g, 52.0 mmol), bis(pinacolato)diboron (15.9 g, 62.5 mmol), toluene (170 mL), DMF (102 mL), and the mixture was degassed by sparging the solution with nitrogen gas, with agitation, for a period of about 15 min. To the solution was added potassium acetate (15.3 g, 156.2 mmol) and Pd(dppf)Cl₂·CH₂Cl₂ (2.12 g, 2.6 mmol), and the mixture was degassed by sparging the solution with nitrogen gas for a period of about 15 min. The reaction solution was heated to a temperature of about 95° C. to 100° C. and agitated at this temperature for no less than 4 h until completion of the reaction. Reaction completion can be assessed by HPLC, wherein the reaction is deemed complete if no more than 1% of compound D remains. The reaction mixture was adjusted to a temperature of about 50° C., then a 5 wt. % solution of N-acetyl cysteine (170 mL) was added. The reaction mixture was cooled to a temperature of about 20° C. to 30° C. and agitated for 1 h, and the layers were allowed to settle. The layers were separated, and the organic layer was washed with 10 wt. % NaCl (2×170 mL). Activated carbon (8.5 g) was charged to the organic layer, the mixture was agitated for about 1.5 h, and the suspension was filtered through celite. The filtrate was evaporated and solvent swapped with 2-propanol (3×85 mL). The volume was adjusted to about 90 mL, and the solution was agitated at a temperature of about 20° C. to 25° C. for 1 h. Resulting suspension was allowed to cool to a temperature of about 0° C. to 5° C. and agitate for 1 h. The resultant solids were filtered, washed with cold 2-propanol (35 mL) and dried at a temperature of no more than 50° C. to provide the title compound as a brown solid (14.5 g, 76% yield). ¹H NMR (400 MHz, DMSO-d₆): δ 10.29 (s, 1H), 8.19 (d, J=7.6 Hz, 1H), 7.71-7.68 (m, 2H), 7.48 (t, J=7.2 Hz, 1H), 7.41 (d, J=7.2 Hz, 1H), 4.04 (s, 3H), 1.34 (s, 12H). MS (ESI+, m/z): Calc'd for [C₁₉H₂₁BClNO₄+H]⁺: 374.1; Found: 374.1.

### Step 2″—Synthesis of 6-(2-chloro-3-(2,3-dichloropyridin-4-yl)phenyl)-2-methoxynicotinaldehyde

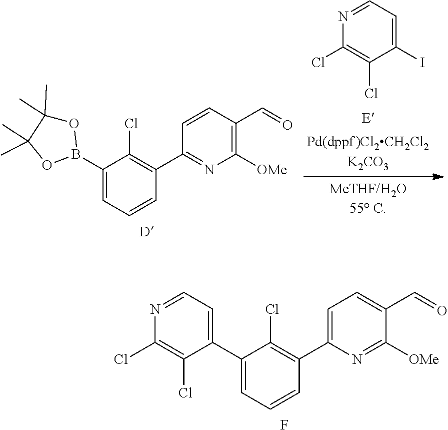

A 1 L reactor was charged with MeTHF (225 mL) and water (49.5 mL), and degassed by sparging the solution with nitrogen gas for a period of about 15 min. Compound E′ (11.0 g, 40.1 mmol) and K₂CO₃ (16.6 g, 120.4 mmol) were added, and the mixture was allowed to agitate and sparged with nitrogen gas for a period of about 15 min. Pd(dppf)Cl₂·CH₂Cl₂ (1.3 g, 1.6 mmol) was added and the mixture was sparged with nitrogen gas for about 15 min. The reaction mixture was heated to a temperature of about 55° C., at which temperature the solution became clear and colorless. In another 250 mL reactor, compound D′ (15.0 g, 40.1 mmol) and MeTHF (75 mL) were added, and the solution was degassed by sparging with nitrogen gas, with agitation, for a period of about 15 min. Next, the solution of compound D′ was slowly added to the 1 L reactor over a period of about 1.5 h, while maintaining the reaction temperature at about 50° C. to 60° C. The reaction mixture was allowed to agitate at 55° C. for no less than 12 h until completion of the reaction. Reaction completion can be assessed by HPLC, wherein the reaction is deemed complete if no more than 2.0% of compound E′ remained. The reaction mixture was adjusted to a temperature of about 50° C. and 5 wt. % N-acetyl cysteine solution (150 mL) was added. The reaction mixture was allowed to cool and agitated at a temperature of about 20° C. to 30° C. for a period of about 1 h. MeTHF (150 mL) and 5 wt % NaCl (75 mL) were added to the reaction mixture. The mixture was agitated for 5 min, then the layers were allowed to settle, and aqueous layer was separated. The organic layer was washed with 10 wt. % NaCl (75 mL), dried over MgSO₄, and filtered. The filtrate was evaporated to a volume of about 45 mL, and solvent swapped into 2-propanol (2×120 mL). Next, additional 2-propanol (75 mL) was added, and the solvent was evaporated until the solution had a total volume of about 105 mL. The resultant suspension was heated to a temperature of about 50° C. to 55° C. and maintained at this temperature for a period of about 1 h. Next, the solution was cooled to a temperature of about 20° C. to 25° C., and agitated for 1 h. The resultant solids were filtered, washed with 2-propanol (30 mL), and dried at a temperature of no more than 55° C. to provide the title compound as a light brown solid (12.5 g, 79% yield).

### Step 3—Synthesis of 2-methoxy-4-(4,4,5,5-tetramethyl-1,3,2-dioxaborolan-2-yl)benzaldehyde

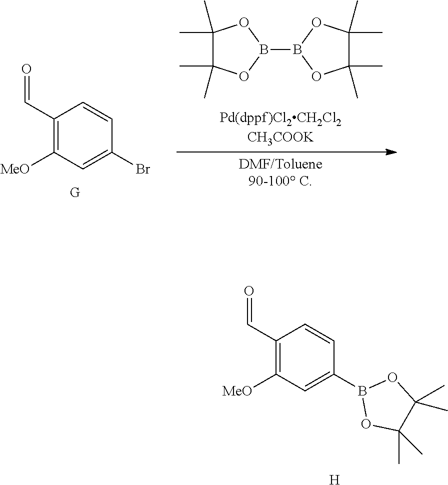

To a 15 L reactor was added compound G (470.0 g, 2.2 mol), bis(pinacolato)diboron (666.0 g, 2.6 mol), potassium acetate (321.7 g, 3.3 mol), toluene (4.7 L) and DMF (2.8 L). The atmosphere of the resulting suspension was evacuated and back filled with nitrogen gas three times, and the suspension was subsequently sparged with nitrogen gas for a period of about 1 h. Next, Pd(dppf)Cl₂ (48.0 g, 65.6 mmol) was added, and the mixture was sparged with nitrogen gas for a period of about 30 min. The reaction mixture was heated to a temperature of about 85° C. and agitated for no less than 7 h until completion of the reaction as determined by HPLC. Reaction completion can be assessed by HPLC, wherein the reaction is deemed complete if no more than 1.0% of compound G remained. The reaction mixture was allowed to cool to 27° C., then a solution of N-acetyl cysteine (4.7 L, 5 wt %) was added, while maintaining an internal temperature of about 23° C. to 31° C. and agitated for 1 h. Next, the phases were separated, and 5 wt % NaCl (4.7 L) was added to the organic layer. The mixture was agitated and filtered through celite. The filtrate was allowed to settle, the phases were separated, and the organic layer was washed with 5 wt % NaCl (4.7 L). Activated carbon (235.0 g) was added to the organic layer and the mixture was agitated for 1.5 h, then filtered through celite. The filtrate was evaporated to a volume of about 1 L and solvent swapped into 2-propanol (2×4.7 L) to provide a solution with a volume of about 1.2 L. The solution was heated to a temperature of about 50° C. and maintained at this temperature for a period of about 1.5 h, then allowed to slowly cool to a temperature of about 6° C. over a period of about 3 h, and agitated at this temperature for a period of about 1.5 h. The resultant solids were filtered, washed with 2-propanol (2×0.45 L) and dried at a temperature of no more than 50° C. for 12 h to provide the title compound as a light-brown solid (492.0 g, 86% yield). ¹H NMR (400 MHz, CDCl₃) δ 10.50 (s, 1H), 7.80 (d, J=7.6 Hz, 1H), 7.45 (dd, J=7.6, 0.8 Hz, 1H), 7.39 (s, 1H), 3.97 (s, 3H), 1.35 (s, 12H). MS (ESI+, m/z): Calc'd for [C₁₄H₂₀BO₄+H]⁺: 263.15; Found: 263.2.

### Step 4—Synthesis of 6-(2-chloro-3-(3-chloro-2-(4-formyl-3-methoxyphenyl)pyridin-4-yl)phenyl)-2-methoxynicotinaldehyde

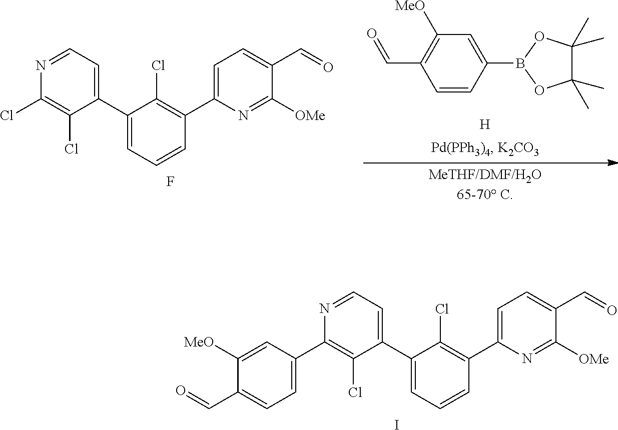

To a 5 L reactor was added K₂CO₃ (526.6 g, 3.8 mol) and water (2.0 L), and the mixture was agitated to facilitate dissolution of solids. The atmosphere of the solution in was evacuated and back filled with nitrogen gas four times, and the solution was subsequently degassed by sparging the solution with nitrogen gas for a period of about 2 h. To a separate 30 L reactor was added MeTHF (7.5 L), DMF (2.0 L), compound F (500.0 g, 1.27 mol), and compound H (349.6 g, 1.33 mol), and the mixture was agitated to facilitate dissolution of solids. The atmosphere of the solution was evacuated and back filled with nitrogen gas four times, and the solution was subsequently degassed by sparging the solution with nitrogen gas for a period of about 1 h. Next, the K₂CO₃ solution was added to the reaction solution followed by Pd(PPh₃)₄ (44.0 g, 0.04 mol), and the resultant mixture was sparged with nitrogen for a period of about 1 h. The reaction mixture was heated to a temperature of about 65° C. to 70° C. and agitated at this temperature for no less than 3 h until completion of the reaction. Reaction completion can be assessed by HPLC, wherein the reaction is deemed complete if no more than 1.0% of compound F remains. The reaction mixture was adjusted to a temperature of about 60° C., then a 5 wt. % N-acetyl cysteine solution (5.0 L) was slowly added over a period of about 40 min. The reaction mixture was allowed to cool to a temperature of about 22° C. over a period of about 1.5 h and allowed to agitate at this temperature for 1.5 h. The resulting solids were filtered, washed with water (7.5 L), and washed with EtOH (2.5 L). The wet cake was dried at a temperature of about 50° C. to provide the title compound as a tan solid (581 g, 92% yield). ¹H NMR (400 MHz, CDCl₃) δ 10.53 (s, 1H), 10.42 (s, 1H), 8.69 (d, J=4.8, 1H), 8.20 (d, J=7.7, 1H), 7.93 (d, J=8.0 Hz, 1H), 7.74 (dd, J=7.7, 1.7 Hz, 1H), 7.52 (t, J=7.7 Hz, 1H), 7.47-7.34 (m, 4H), 7.32 (d, J=4.8 Hz, 1H), 4.13 (s, 3H), 4.00 (d, 3H). MS (ESI+, m/z): Calc'd for [C₂₆H₁₉C₁₂N₂O₄+H]⁺: 493.07; Found: 493.1.

### Step 5—Synthesis of 2-((6-(2-chloro-3-(3-chloro-2-(3-methoxy-4-((7-oxo-2,6-diazaspiro[3.4]octan-2-yl)methyl)phenyl)pyridin-4-yl)phenyl)-2-methoxypyridin-3-yl)methyl)-2,6-diazaspiro[3.4]octan-7-one

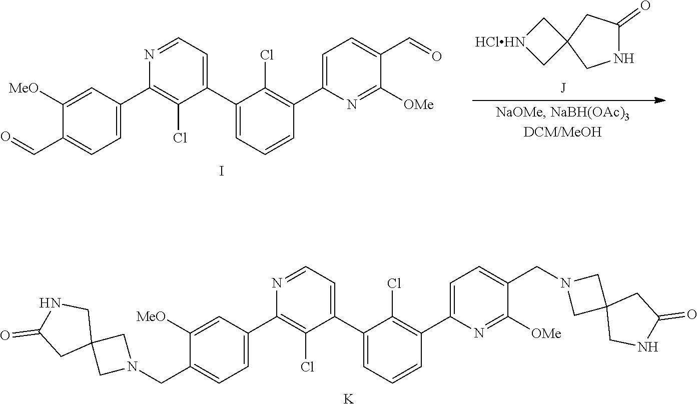

To a 50 L reactor was added compound J (332.3 g, 2.04 mol), CH₂Cl₂ (13.4 L) and MeOH (2.9 L), and the mixture was allowed to agitate. Next, NaOMe (110 g, 2.04 mol, 25 wt. % solution in MeOH) was added and the resulting slurry was agitated at a temperature of about 15° C. to 20° C. for 30 min. Compound I (420.0 g, 0.851 mol) was added, and the mixture was agitated at a temperature of about 22° C. to 28° C. for 1 h. The slurry was cooled to a temperature of about 18° C. and NaBH(OAc)₃ (1.08 kg, 5.1 mol) was added portion-wise (i.e., 3 portions, wherein each portion is added at an interval of about 20 mins). The reaction mixture was stirred for no less than 1 h, until completion of the reaction as determined. Reaction completion can be assessed by HPLC, wherein the reaction is deemed complete if no more than 1.0% of compound I remains. To the reaction mixture was added water (8.4 L), and the resulting clear biphasic mixture was agitated at a temperature of about 20° C. to 25° C. for a period of about 20 min. The layers were allowed to settle and were separated. The aqueous layer as washed with CH₂Cl₂ (6.3 L). To the aqueous phase was added CH₂Cl₂ (8.4 L), and the pH of the aqueous layer was adjusted to pH 8-9 using 3 N aqueous NaOH (3.2 L). The resultant mixture was agitated for 20 min, then the phases were separated. The aqueous layer was further extracted with CH₂Cl₂ (2.1 L) and the combined organic layers were washed with water (4.2 L). The aqueous layer was back extracted with CH₂Cl₂ (2.1 L) and the combined organic layers were filtered through a pad of Na₂SO₄. The filtered solution was concentrated in vacuo and solvent swapped with 2-propanol (2×2.1 L) at a temperature of about 40° C. to 50° C. The final volume of the solution was adjusted to a volume of about 2.1 L. The solution was seeded with compound K (2.1 g) to provide a fine slurry, which was agitated at a temperature of about 50° C. for a period of about 2 h, then gradually cooled to a temperature of about 20° C. over a period of about 10 h. The resulting thick slurry was stirred at the same temperature (i.e., about 20° C.) for a period of about 4 h. The solids were filtered and washed with 2-propanol (840 mL). The wet cake was dried in vacuo at 55° C. for no less than 24 h to provide the title compound as a white to off-white solid (533 g, 88% yield). ¹H NMR (400 MHz, CDCl₃) δ: 8.63 (d, J=4.8 Hz, 1H), 7.71 (dd, J=1.7, 7.7 Hz, 1H), 7.62 (d, J=7.5 Hz, 1H), 7.46 (t, J=7.7 Hz, 1H), 7.38-7.31 (m, 2H), 7.30 (dd, J=1.7, 7.7 Hz, 1H), 7.28-7.23 (m, 3H), 5.74 (br s, 1H), 5.68 (br s, 1H), 4.00 (s, 3H), 3.88 (s, 3H), 3.70 (s, 2H), 3.65 (s, 2H), 3.61 (s, 2H), 3.58 (s, 2H), 3.40-3.28 (m, 8H), 2.56 (s, 2H), 2.54 (s, 2H); MS (ESI+, m/z): Calc'd for [C₃₈H₃₈C₁₂N₆O₄+H]⁺: 713.2; Found: 713.2.

In certain embodiments, the recrystallization described herein utilizes a seed crystal of compound K to facilitate recrystallization. In certain embodiments, the seed crystal of compound K is prepared as described in Step 5, except that recrystallization is not facilitated by the addition of a seed crystal.

### Step 5′—Synthesis of 2-((6-(2-chloro-3-(3-chloro-2-(3-methoxy-4-((7-oxo-2,6-diazaspiro[3.4]octan-2-yl)methyl)phenyl)pyridin-4-yl)phenyl)-2-methoxypyridin-3-yl)methyl)-2,6-diazaspiro[3.4]octan-7-one

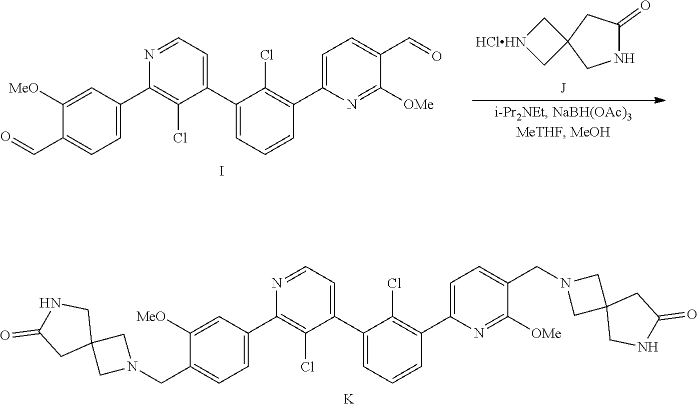

To a 50 L reactor was added compound J (553.8 g, 3.40 mol), MeTHF (21 L) and MeOH (7 L), and the mixture was allowed to agitate. DIPEA (458.5 g, 3.5 mol) was added and the resulting slurry was agitated at a temperature of about 15° C. to 20° C. for 1 h. Compound I (700.0 g, 1.42 mol) was added, and the mixture was agitated at 33° C. to 35° C. for 1.5 h. The slurry was cooled to a temperature of about 15° C. to 20° C. and NaBH(OAc)₃ (1.8 kg, 8.5 mol) was added portion-wise (i.e., 4 portions, wherein each portion is added at an interval of about 20 mins). The reaction mixture was allowed to warm to 20° C. to 25° C. and agitated for no less than 1 h, until completion of the reaction. Reaction completion can be assessed by HPLC, wherein the reaction is deemed complete if no more than 1.0% of compound I remains. To the reaction mixture was added 10% NaCl (9 L), and the resulting clear biphasic mixture was agitated at a temperature of about 20° C. to 25° C. for a period of about 20 min. The layers were allowed to settle and were separated. The organic layer was extracted with 10% NaCl (3.5 L). The combined aqueous layer was sequentially washed with MeTHF (3.5 L) and MEK (2×7 L). To the aqueous phase was added MEK (14 L), and the pH of the aqueous layer was adjusted to pH 9.5-10.5 using 20 wt % aqueous Na₂CO₃ (9 L). The resultant mixture was agitated for 20 min, then the phases were separated. The aqueous layer was further extracted with MEK (2×9 L). The combined organic layers were washed with 24 wt % NaCl (7 L). The organic layer was filtered through a pad of Na₂SO₄. The filtered solution was diluted with MeOH (3.5 L) and was concentrated in vacuo and solvent swapped with three portions of MeOH (17 L) at a temperature of about 40° C. to 50° C. The final volume of the solution was adjusted to a volume of about 3.5 L. The solution was allowed to agitate at 48° C. to 50° C. and was seeded with compound K (3.5 g) to provide a fine slurry, which was agitated at a temperature of about 50° C. for a period of about 3 h, then gradually cooled to a temperature of about 20° C. over a period of about 4 h. The resulting thick slurry was agitated at the same temperature (i.e., about 20° C.) for a period of about 10 h. MTBE (7 L) was charged and the slurry was allowed to stir for 4 h. The solids were filtered and washed with 30% MeOH/MTBE (2×1.4 L). The wet cake was dried in vacuo at 65° C. for no less than 24 h to provide the title compound as a white to off-white solid (760 g, 74% yield).

In certain embodiments, compound K obtained by the method described herein may contain higher levels of residual MTBE. Excess MTBE may be reduced by preparing a slurry of the isolated material in hot MeOH, cooling the slurry to ambient temperature, filtering, washing with MeOH, and drying. In certain embodiments, the recrystallization described herein utilizes solvents other than MTBE as an anti-solvent.

### Enumerated Embodiments

The following exemplary embodiments are provided, the numbering of which is not to be construed as designating levels of importance:

Embodiment 1 provides a method of preparing 2-((6-(2-chloro-3-(3-chloro-2-(3-methoxy-4-((7-oxo-2,6-diazaspiro[3.4]octan-2-yl)methyl)phenyl)pyridin-4-yl)phenyl)-2-methoxypyridin-3-yl)methyl)-2,6-diazaspiro[3.4]octan-7-one (K), or a salt or solvate thereof:

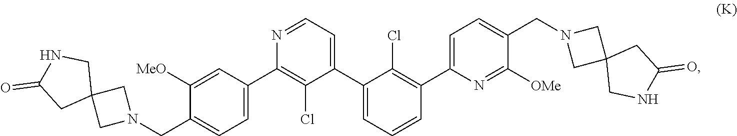 [SMILES: COc1cc(-c2nccc(-c3cccc(-c4ccc(CN5CC6(CNC(=O)C6)C5)c(OC)n4)c3Cl)c2Cl)ccc1CN1CC2(CNC(=O)C2)C1]

the method comprising reacting 6-(2-chloro-3-(3-chloro-2-(4-formyl-3-methoxyphenyl)pyridin-4-yl)phenyl)-2-methoxynicotinaldehyde (I):

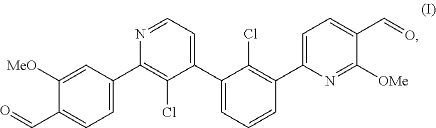 [SMILES: COc1cc(-c2nccc(-c3cccc(-c4ccc(C=O)c(OC)n4)c3Cl)c2Cl)ccc1C=O]

and 2,6-diazaspiro[3.4]octan-7-one (J):

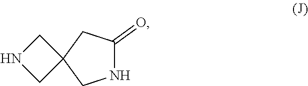

in the presence of a reducing agent and a base, so as to generate a first reaction system comprising (K).

Embodiment 2 provides the method of Embodiment 1, wherein the reducing agent is NaBH(OAc)₃.

Embodiment 3 provides the method of Embodiment 1 or 2, wherein the base comprises NaOMe or i-PrNEt₂.

Embodiment 4 provides the method of any one of Embodiments 1-3, wherein (J) is selected from the group consisting of 2,6-diazaspiro[3.4]octan-7-one hydrochloride, 2,6-diazaspiro[3.4]octan-7-one hydrobromide, 2,6-diazaspiro[3.4]octan-7-one trifluoroacetate, 2,6-diazaspiro[3.4]octan-7-one mesylate, and 2,6-diazaspiro[3.4]octan-7-one tosylate.

Embodiment 5 provides the method of any one of Embodiments 1-4, wherein the reaction of (I) and (J) occurs in the presence of a solvent.

Embodiment 6 provides the method of Embodiment 5, wherein the solvent comprises at least one of a mixture comprising dichloromethane (DCM) and methanol (MeOH), a mixture comprising 2-methyltetrahydrofuran (MeTHF) and MeOH, tetrahydrofuran (THF), dimethylformamide (DMF), and dimethylacetamide (DMAc), or any mixtures thereof.

Embodiment 7 provides the method of any one of Embodiments 1-6, wherein purification of (K) comprises:
(a) adding water to the first reaction system comprising (K) to generate a biphasic solution;
(b) separating the biphasic solution to provide a first aqueous phase and a first organic phase;
(c) adding an organic solvent to the first aqueous phase to provide a second biphasic solution;
(d) basifying the second biphasic solution to pH 8-11 to provide a basified biphasic solution; and
(e) separating the basified biphasic solution to provide a second aqueous phase and a second organic phase comprising (K).

Embodiment 8 provides the method of any one of Embodiments 1-7, wherein purification of (K) comprises:
(a) providing crude (K) in a solvent comprising 2-propanol to afford a dilute crude solution of (K),
   wherein the dilute crude solution of (K) has a concentration of about 110 g/L to about 140 g/L;
(b) at least partially evaporating the dilute crude solution of (K) to provide a concentrated crude solution of (K),
   wherein the concentrated crude solution of (K) has a concentration of about 220 g/L to about 280 g/L;
(c) cooling the concentrated crude solution of (K) to a temperature of about 20° C. provide a purified (K) slurry; and
(d) filtering the purified slurry of (K) to provide (K).

Embodiment 9 provides the method of any one of Embodiments 1-7, wherein purification of (K) comprises:
(a) providing crude (K) in a solvent comprising MeOH to afford a crude solution of (K),
   wherein the crude solution of (K) has a concentration of about 200 g/L to about 300 g/L;
(b) heating the crude solution of (K) to a temperature of about 50° C. to provide a hot crude solution of (K);
(c) cooling the hot crude solution of (K) to a temperature of about 20° C. provide a cooled (K) slurry;
(d) adding a solvent comprising methyl tert-butyl ether (MTBE) to the cooled (K) slurry to provide a purified (K) slurry; and
(e) filtering the purified (K) slurry to provide (K).

Embodiment 10 provides the method of any one of Embodiments 1-9, wherein (I) is prepared by reacting 2-methoxy-4-(B(OR^{1a})(OR^{1b}))-benzaldehyde (H):

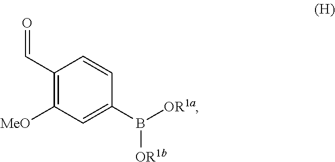

and 6-(2-chloro-3-(2,3-dichloropyridin-4-yl)phenyl)-2-methoxynicotinaldehyde (F):

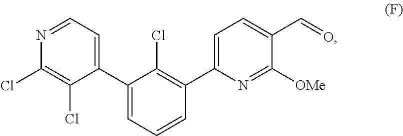 [SMILES: COc1nc(-c2cccc(-c3ccnc(Cl)c3Cl)c2Cl)ccc1C=O]

in the presence of a palladium catalyst and a base;
   wherein each R^{1a} and R^{1b} are each independently selected from the group consisting of C₁-C₆ alkyl and C₃-C₈ cycloalkyl,
      or R^{1a} and R^{1b} may combine with the atoms to which they are bound to form a C₂-C₃ heterocycloalkyl.

Embodiment 11 provides the method of Embodiment 10, wherein the palladium catalyst comprises Pd(PPh₃)₄.

Embodiment 12 provides the method of Embodiment 10 or 11, wherein the palladium catalyst is present in an amount ranging from about 0.1 mol % to about 5 mol %.

Embodiment 13 provides the method of any one of Embodiments 10-12, wherein the base comprises K₂CO₃.

Embodiment 14 provides the method of any one of Embodiments 10-13, wherein the reaction of (H) and (F) occurs in the presence of a solvent.

Embodiment 15 provides the method of Embodiment 14, wherein the solvent comprises at least one of 2-methyltetrahydrofuran (MeTHF), dimethylformamide (DMF), and water.

Embodiment 16 provides the method of any one of Embodiments 10-15, wherein the reaction of (H) and (F) occurs at a temperature of about 65° C. to about 70° C.

Embodiment 17 provides the method of any one of Embodiments 10-16, wherein purification of (I) comprises adding a N-acetyl cysteine to the reaction of (H) and (F).

Embodiment 18 provides the method of any one of Embodiments 10-17, wherein (H) is prepared by reacting 4-Z²-2-methoxybenzaldehyde (G):

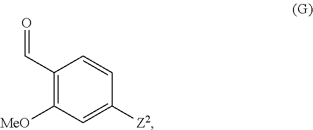

wherein Z² is selected from the group consisting of Cl, Br, and I; and a borylating reagent in the presence of a palladium catalyst and a base.

Embodiment 19 provides the method of Embodiment 18, wherein the borylating reagent comprises bis(pinacolato)diboron.

Embodiment 20 provides the method of Embodiment 18 or 19, wherein the palladium catalyst comprises Pd(dppf)Cl₂.

Embodiment 21 provides the method of any one of Embodiments 18-20, wherein the palladium catalyst is present in an amount ranging from about 0.1 mol % to about 5.0 mol %.

Embodiment 22 provides the method of any one of Embodiments 18-21, wherein the base comprises KOAc.

Embodiment 23 provides the method of any one of Embodiments 18-22, wherein the reaction of (G) and the borylating reagent occurs in the presence of a solvent.

Embodiment 24 provides the method of Embodiment 23, wherein the solvent comprises dimethylformamide (DMF) or toluene.

Embodiment 25 provides the method of any one of Embodiments 18-24, wherein the reaction of (G) and the borylating reagent occurs at a temperature of about 80° C. to about 90° C.

Embodiment 26 provides the method of any one of Embodiments 18-25, wherein purification of (H) comprises at least one of:
(a) adding N-acetyl cysteine to the reaction of (G) and the borylating reagent; and
(b) adding activated carbon to the reaction of (G) and the borylating reagent.

Embodiment 27 provides the method of any one of Embodiments 18-26, wherein purification of (H) comprises:
(a) providing crude (H) in a solvent comprising 2-propanol to afford a dilute crude solution of (H),
   wherein the dilute crude solution of (H) has a concentration of about 390 g/L to about 430 g/L;
(b) at least partially evaporating the dilute crude solution of (H) to provide a concentrated crude solution of (H), wherein the at least partial evaporation optionally comprises heating the dilute crude solution of (H) to a temperature of about 50° C., and further optionally maintaining the temperature for a period of about 1.5 h;
(c) cooling the concentrated crude solution of (H) to a temperature of about 6° C., provide a purified (H) slurry, wherein the cooling optionally occurs over a period of about 3 h; and
(d) filtering the purified (H) slurry to provide (H).

Embodiment 28 provides the method of any one of Embodiments 10-17, wherein (F) is prepared by reacting 6-(3-Z¹-2-chlorophenyl)-2-methoxynicotinaldehyde (D):

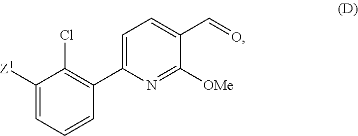

wherein Z¹ is selected from the group consisting of Br and I; and 2,3-dichloro-4-(B(OR^{2a})(OR^{2b}))-pyridine (E):

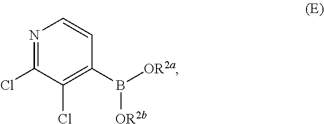

in the presence of a palladium catalyst and a base;
wherein each R^{2a} and R^{2b} are each independently selected from the group consisting of C₁-C₆ alkyl and C₃-C₈ cycloalkyl,
   or R^{2a} and R^{2b} may combine with the atoms to which they are bound to form a C₂-C₃ heterocycloalkyl.

Embodiment 29 provides the method of Embodiment 28, wherein R^{2a} and R^{2b} are each independently H.

Embodiment 30 provides the method of Embodiment 28 or 29, wherein the palladium catalyst comprises Pd(amphos)Cl₂.

Embodiment 31 provides the method of any one of Embodiments 28-30, wherein the palladium catalyst is present in an amount ranging from about 0.1 mol % to about 5 mol %.

Embodiment 32 provides the method of any one of Embodiments 28-31, wherein the base comprises K₂HPO₄.

Embodiment 33 provides the method of any one of Embodiments 28-32, wherein the reaction of (D) and (E) comprises addition of (E) to a reaction vessel comprising (D),
wherein (E) is optionally added to the vessel comprising (D) over a period of about 3.5 h.

Embodiment 34 provides the method of any one of Embodiments 28-33, wherein the reaction of (D) and (E) occurs in the presence of a solvent.

Embodiment 35 provides the method of Embodiment 34, wherein the solvent comprises 2-methyltetrahydrofuran (MeTHF), dimethylacetamide (DMAc), and/or water.

Embodiment 36 provides the method of any one of Embodiments 28-35, wherein the reaction of (D) and (E) occurs at a temperature of about 65° C. to about 70° C.

Embodiment 37 provides the method of any one of Embodiments 28-36, wherein purification of (F) comprises adding a N-acetyl cysteine to the reaction of (D) and (E).

Embodiment 38 provides the method of any one of Embodiments 28-37, wherein purification of (F) comprises:
(a) providing crude (F) in a solvent comprising 2-propanol to afford a dilute crude solution of (F),
   wherein the dilute crude solution of (F) has a concentration of about 100 g/L to about 150 g/L;
(b) at least partially evaporating the dilute crude solution of (F) to provide a concentrated crude solution of (F), wherein the at least partial evaporation optionally comprises heating the dilute crude solution of (F) to a temperature of about 40° C. to about 55° C., and further optionally maintaining the temperature for a period of about 1 h;
(c) cooling the concentrated crude solution of (F) to a temperature of about 20° C. to about 25° C., to provide a crude (F) slurry, wherein the cooling optionally occurs over a period of about 3 h; and
(d) filtering the crude (F) slurry to provide (F), wherein the filtering optionally further comprises washing with a solvent comprising 2-propanol.

Embodiment 39 provides the method of any one of Embodiments 10-17, wherein (F) is prepared by reacting 6-(2-chloro-3-(B(OR^{3a})(OR^{3b}))phenyl)-2-methoxynicotinaldehyde (D′):

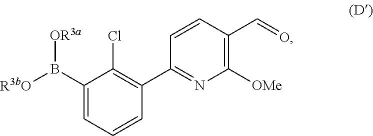

and 2,3-dichloro-4-Zi-pyridine (E′):

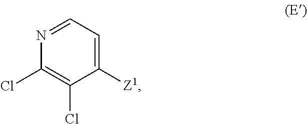

wherein Z¹ is selected from the group consisting of Br and I; in the presence of a palladium catalyst and a base;
wherein each R^{3a} and R^{3b} are each independently selected from the group consisting of C₁-C₆ alkyl and C₃-C₈ cycloalkyl,
or R^{3a} and R^{3b} may combine with the atoms to which they are bound to form a C₂-C₃ heterocycloalkyl.

Embodiment 40 provides the method of Embodiment 39, wherein (D′) is 6-(2-chloro-3-(4,4,5,5-tetramethyl-1,3,2-dioxaborolan-2-yl)phenyl)-2-methoxynicotinaldehyde;

Embodiment 41 provides the method of Embodiment 39 or 40, wherein the palladium catalyst comprises Pd(dppf)Cl₂.

Embodiment 42 provides the method of any one of Embodiments 39-41, wherein the palladium catalyst is present in an amount ranging from about 0.1 mol % to about 5 mol %.

Embodiment 43 provides the method of any one of Embodiments 39-42, wherein the base comprises K₂CO₃.

Embodiment 44 provides the method of any one of Embodiments 39-43, wherein the reaction of (D′) and (E′) occurs in the presence of a solvent.

Embodiment 45 provides the method of Embodiment 44, wherein the solvent comprises 2-methyltetrahydrofuran (MeTHF) and water.

Embodiment 46 provides the method of any one of Embodiments 39-45, wherein the reaction of (D′) and (E′) occurs at a temperature of about 55° C.

Embodiment 47 provides the method of any one of Embodiments 39-46, wherein purification of (F) comprises adding N-acetyl cysteine to the reaction of (D′) and (E′).

Embodiment 48 provides the method of any one of Embodiments 39-47, wherein purification of (F) comprises:
(a) providing crude (F) in 2-propanol to afford a dilute crude solution of (F),
   wherein the dilute crude solution of (F) has a concentration of about 100 g/L to about 140 g/L;
(b) at least partially evaporating the dilute crude solution of (F) to provide a concentrated crude solution of (F), wherein the at least partial evaporation optionally comprises heating the dilute crude solution of (F) to a temperature of about 50° C. to about 55° C., and further optionally maintaining the temperature for a period of about 1 h;
(c) cooling the concentrated crude solution of (F) to a temperature of about 20° C. to about 25° C., to provide a purified (F) slurry; and
(d) filtering the purified (F) slurry to provide (F).

Embodiment 49 provides the method of any one of Embodiments 39-48, wherein (D′) is prepared by reacting (D):

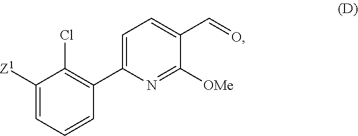

wherein Z¹ is selected from the group consisting of Br and I; and a borylating reagent in the presence of a palladium catalyst and a base.

Embodiment 50 provides the method of Embodiment 49, wherein the borylating reagent comprises bis(pinacolato)diboron.

Embodiment 51 provides the method of Embodiment 49 or 50, wherein the palladium catalyst comprises Pd(dppf)Cl₂.

Embodiment 52 provides the method of any one of Embodiments 49-51, wherein the palladium catalyst is present in an amount ranging from about 0.1 mol % to about 7 mol %.

Embodiment 53 provides the method of any one of Embodiments 49-52, wherein the base comprises KOAc.

Embodiment 54 provides the method of any one of Embodiments 49-53, wherein the reaction of (D) and the borylating reagent occurs in the presence of a solvent.

Embodiment 55 provides the method of Embodiment 54, wherein the solvent comprises dimethylformamide (DMF) and/or toluene.

Embodiment 56 provides the method of any one of Embodiments 49-55, wherein the reaction of (D) and the borylating reagent occurs at temperature of about 90° C. to about 100° C.

Embodiment 57 provides the method of any one of Embodiments 49-56, wherein purification of (D′) comprises at least one of:
(a) adding a N-acetyl cysteine to the reaction of (D) and the borylating reagent; and
(b) adding activated carbon to the reaction of (D) and the borylating reagent.

Embodiment 58 provides the method of any one of Embodiments 49-57, wherein purification of (D′) comprises:
(a) providing crude (D′) in a solvent comprising 2-propanol to afford a crude solution of (D′),
   wherein the crude solution of (D′) has a concentration of about 140 g/L to about 180 g/L;
(c) cooling the crude solution of (D′) to a temperature of about 0° C. to about 5° C., to provide a purified (D′) slurry; and
(d) filtering the purified (D′) slurry to provide (D′).

Embodiment 59 provides the method of any one of Embodiments 28-38 and 49-58, wherein (D) is prepared by reacting 1-(B(OR^{4a})(OR^{4b}))-2-chloro-3-Z¹-benzene (B):

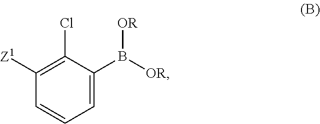

and 6-Z²-2-methoxynicotinaldehyde (C):

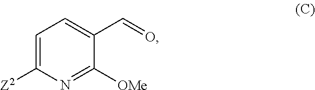

in the presence of a palladium catalyst and a base;
   wherein Z¹ is selected from the group consisting of Br and I;
   wherein Z² is selected from the group consisting of Cl, Br, and I;
   wherein each R^{4a} and R^{4b} are each independently selected from the group consisting of C₁-C₆ alkyl and C₃-C₈ cycloalkyl,
      or R^{4a} and R^{4b} may combine with the atoms to which they are bound to form a C₂-C₃ heterocycloalkyl.

Embodiment 60 provides the method of Embodiment 59, wherein (B) is 2-(3-bromo-2-chlorophenyl)-4,4,5,5-tetramethyl-1,3,2-dioxaborolane.

Embodiment 61 provides the method of Embodiment 59 or 60, wherein the palladium catalyst comprises Pd(PPh₃)₄.

Embodiment 62 provides the method of any one of Embodiments 59-61, wherein the palladium catalyst is present in an amount ranging from about 0.1 mol % to about 5 mol %.

Embodiment 63 provides the method of any one of Embodiments 59-62, wherein the base comprises K₂CO₃.

Embodiment 64 provides the method of any one of Embodiments 59-63, wherein the reaction of (B) and (C) occurs in the presence of a solvent.

Embodiment 65 provides the method of Embodiment 64, wherein the solvent comprises 2-methyltetrahydrofuran (MeTHF) and/or water.

Embodiment 66 provides the method of any one of Embodiments 59-65, wherein the reaction of (B) and (C) occurs at a temperature of about 50° C. to about 60° C.

Embodiment 67 provides the method of any one of Embodiments 59-66, wherein purification of (D) comprises adding N-acetyl cysteine to the reaction of (B) and (C).

Embodiment 68 provides the method of any one of Embodiments 59-67, wherein purification of (D) comprises:
(a) providing crude (D) in solvent comprising 2-propanol to afford a dilute crude solution of (D),
   wherein the dilute crude solution of (D) has a concentration of about 160 g/L to about 220 g/L;
(b) at least partially evaporating the dilute crude solution of (D) to provide a concentrated crude solution of (D), wherein the at least partial evaporation optionally comprises heating the dilute crude solution of (D) to a temperature of about 50° C. to about 55° C., and further optionally maintaining the temperature for a period of about 1 h;
(c) cooling the concentrated crude solution of (D) to a temperature of about 20° C. to about 25° C., to provide a purified (D) slurry; and
(d) filtering the purified (D) slurry to provide (D).

Embodiment 69 provides the method of any one of Embodiments 59-68, wherein (B) is prepared by:
(a) reacting 1-Z¹-2-chloro-3-Z¹-benzene (A):

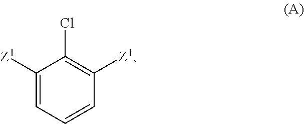

wherein each occurrence of Z¹ is independently selected from the group consisting of Br and I;
and an organomagnesium halide to form an arylmagnesium intermediate; and
(b) reacting the arylmagnesium intermediate with a borate.

Embodiment 70 provides the method of Embodiment 69, wherein the borate is:

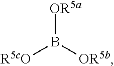

wherein each R^{5a}, R^{5b}, and R^{5c} are each independently selected from the group consisting of C₁-C₆ alkyl and C₃-C₈ cycloalkyl,
   or any two selected from the group consisting of R^{5a}, R^{5b}, and R^{5c} may combine with the atoms to which they are bound to form a C₂-C₃ heterocycloalkyl.

Embodiment 71 provides the method of Embodiment 69 or 70, wherein the borate comprises 2-isopropoxy-4,4,5,5-tetramethyl-1,3,2-dioxaborolane.

Embodiment 72 provides the method of any one of Embodiments 69-71, wherein the organomagnesium halide comprises i-PrMgCl, wherein the i-PrMgCl is optionally a solution comprising i-PrMgCl complexed with LiCl (i.e., an i-PrMgCl·LiCl solution).

Embodiment 73 provides the method of any one of Embodiments 69-72, wherein the reaction of (A) and the organomagnesium halide occurs in a solvent.

Embodiment 74 provides the method of Embodiment 73, wherein the solvent comprises 2-methyltetrahydrofuran (MeTHF).

Embodiment 75 provides the method of any one of Embodiments 69-74, wherein the reaction of (A) and the organomagnesium halide occurs at a temperature of about −25° C. to about −15° C.

Embodiment 76 provides the method of any one of Embodiments 69-75, wherein the reaction of the arylmagnesium intermediate and the borate occurs at a temperature of about −18° C. to about −15° C., wherein the reaction is optionally subsequently warmed to a temperature of about 2° C.

Embodiment 77 provides the method of any one of Embodiments 28-38 and 49-58, wherein (D) is prepared by:
(a) providing 1-Z¹-2-chloro-3-Z¹-benzene (A):

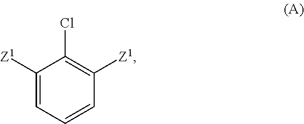

wherein each occurrence of Z¹ is independently selected from the group consisting of Br and I;
and an organomagnesium halide to form an arylmagnesium intermediate;
(b) reacting the arylmagnesium intermediate with a borate to provide a boronic ester intermediate; and
(c) reacting the boronic ester intermediate and 6-Z²-2-methoxynicotinaldehyde (C):

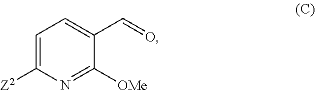

wherein Z² is selected from the group consisting of Cl, Br, and I;
in the presence of a palladium catalyst and a base.

Embodiment 78 provides the method of Embodiment 77, wherein the organomagnesium halide comprises i-PrMgCl, wherein the i-PrMgCl is optionally a solution comprising i-PrMgCl complexed with LiCl (i.e., an i-PrMgCl·LiCl solution).

Embodiment 79 provides the method of Embodiment 77 and 78, wherein the reaction of (A) and the organomagnesium halide occurs in a solvent.

Embodiment 80 provides the method of Embodiment 79, wherein the solvent comprises 2-methyltetrahydrofuran (MeTHF).

Embodiment 81 provides the method of any one of Embodiments 77-80, wherein the reaction of (A) and the organomagnesium halide occurs at a temperature of about −25° C. to about −15° C.

Embodiment 82 provides the method of any one of Embodiments 77-81, wherein the reaction of the arylmagnesium intermediate and the borate occurs at a temperature of about −18° C. to about −15° C., wherein the reaction is optionally subsequently warmed to a temperature of about 2° C.

Embodiment 83 provides the method of any one of Embodiments 77-82, wherein the borate is:

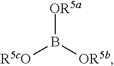

wherein each R^{5a}, R^{5b}, and R^{5c} are each independently selected from the group consisting of C₁-C₆ alkyl and C₃-C₈ cycloalkyl,
   or any two selected from the group consisting of R^{5a}, R^{5b}, and R^{5c} may combine with the atoms to which they are bound to form a C₂-C₃ heterocycloalkyl.

Embodiment 84 provides the method of any one of Embodiments 77-83, wherein the borate comprises 2-isopropoxy-4,4,5,5-tetramethyl-1,3,2-dioxaborolane.

Embodiment 85 provides the method of any one of Embodiments 77-84, wherein the palladium catalyst comprises Pd(PPh₃)₄.

Embodiment 86 provides the method of any one of Embodiments 77-85, wherein the palladium catalyst is present in an amount ranging from about 0.1 mol % to about 5 mol %.

Embodiment 87 provides the method of any one of Embodiments 77-86, wherein the base comprises K₂CO₃.

Embodiment 88 provides the method of any one of Embodiments 77-87, wherein the reaction of the boronic ester intermediate and (C) occurs in the presence of a solvent.

Embodiment 89 provides the method of Embodiment 88, wherein the solvent comprises 2-methyltetrahydrofuran (MeTHF) and/or water.

Embodiment 90 provides the method of any one of Embodiments 77-89, wherein the reaction of the boronic ester intermediate and (C) occurs at a temperature of about 50° C. to about 60° C.

Embodiment 91 provides the method of any one of Embodiments 77-90, wherein purification of (D) comprises adding N-acetyl cysteine to the reaction of (C) and the boronic ester intermediate.

Embodiment 92 provides the method of any one of Embodiments 77-91, wherein purification of (D) comprises:
(a) providing crude (D) in a solvent comprising 2-propanol to afford a dilute crude solution of (D),
   wherein the dilute crude solution of (D) has a concentration of about 160 g/L to about 220 g/L;
(b) at least partially evaporating the dilute crude solution of (D) to provide a concentrated crude solution of (D), wherein the at least partial evaporation optionally comprises heating the dilute crude solution of (D) to a temperature of about 50° C. to about 55° C., and further optionally maintaining the temperature for a period of about 1 h;
(c) cooling the concentrated crude solution of (D) to a temperature of about 20° C. to about 25° C., to provide a purified (D) slurry; and
(d) filtering the purified (D) slurry to provide (D).

The terms and expressions employed herein are used as terms of description and not of limitation, and there is no intention in the use of such terms and expressions of excluding any equivalents of the features shown and described or portions thereof, but it is recognized that various modifications are possible within the scope of the embodiments of the present application. Thus, it should be understood that although the present application describes specific embodiments and optional features, modification and variation of the compositions, methods, and concepts herein disclosed may be resorted to by those of ordinary skill in the art, and that such modifications and variations are considered to be within the scope of embodiments of the present application.

1. A method of preparing 2-((6-(2-chloro-3-(3-chloro-2-(3-methoxy-4-((7-oxo-2,6-diazaspiro[3.4]octan-2-yl)methyl)phenyl)pyridin-4-yl)phenyl)-2-methoxypyridin-3-yl)methyl)-2,6-diazaspiro[3.4]octan-7-one (K), or a salt or solvate thereof: 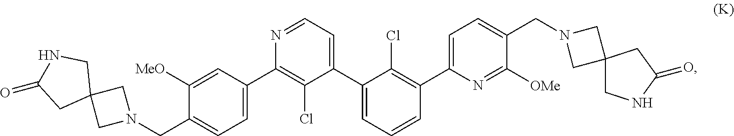 [SMILES: COc1cc(-c2nccc(-c3cccc(-c4ccc(CN5CC6(CNC(=O)C6)C5)c(OC)n4)c3Cl)c2Cl)ccc1CN1CC2(CNC(=O)C2)C1]
the method comprising reacting 6-(2-chloro-3-(3-chloro-2-(4-formyl-3-methoxyphenyl)pyridin-4-yl)phenyl)-2-methoxynicotinaldehyde (I): 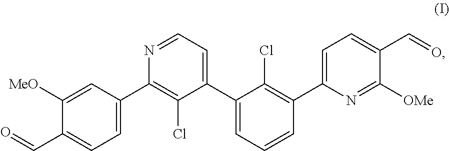 [SMILES: COc1cc(-c2nccc(-c3cccc(-c4ccc(C=O)c(OC)n4)c3Cl)c2Cl)ccc1C=O]
and 2,6-diazaspiro[3.4]octan-7-one (J): 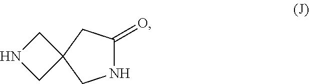
in the presence of a reducing agent and a base, so as to generate a first reaction system comprising (K).

2. The method of claim 1, wherein at least one of the following applies:
(a) the reducing agent is NaBH(OAc)₃,
(b) the base comprises NaOMe or i-PrNEt₂;
(c) (J) is selected from the group consisting of 2,6-diazaspiro[3.4]octan-7-one hydrochloride, 2,6-diazaspiro[3.4]octan-7-one hydrobromide, 2,6-diazaspiro[3.4]octan-7-one trifluoroacetate, 2,6-diazaspiro[3.4]octan-7-one mesylate, and 2,6-diazaspiro[3.4]octan-7-one tosylate; and
(d) the reaction of (I) and (J) occurs in the presence of a solvent, optionally wherein the solvent comprises at least one of a mixture comprising dichloromethane (DCM) and methanol (MeOH), a mixture comprising 2-methyltetrahydrofuran (MeTHF) and MeOH, tetrahydrofuran (THF), dimethylformamide (DMF), and dimethylacetamide (DMAc), or any mixtures thereof.

3-6. (canceled)

7. The method of claim 1, wherein purification of (K) comprises:
(a) adding water to the first reaction system comprising (K) to generate a biphasic solution;
(b) separating the biphasic solution to provide a first aqueous phase and a first organic phase;
(c) adding an organic solvent to the first aqueous phase to provide a second biphasic solution;
(d) basifying the second biphasic solution to pH 8-11 to provide a basified biphasic solution; and
(e) separating the basified biphasic solution to provide a second aqueous phase and a second organic phase comprising (K).

8. The method of claim 1, wherein purification of (K) comprises steps (1-a) to (1-d) or steps (2-a) to (2-e):
(1-a) providing crude (K) in a solvent comprising 2-propanol to afford a dilute crude solution of (K),
wherein the dilute crude solution of (K) has a concentration of about 110 g/L to about 140 g/L;
(1-b) at least partially evaporating the dilute crude solution of (K) to provide a concentrated crude solution of (K),
wherein the concentrated crude solution of (K) has a concentration of about 220 g/L to about 280 g/L;
(1-c) cooling the concentrated crude solution of (K) to a temperature of about 20° C. provide a purified (K) slurry; and
(1-d) filtering the purified slurry of (K) to provide (K);
or
(2-a) providing crude (K) in a solvent comprising MeOH to afford a crude solution of (K), wherein the crude solution of (K) has a concentration of about 200 g/L to about 300 g/L;
(2-b) heating the crude solution of (K) to a temperature of about 50° C. to provide a hot crude solution of (K);
(2-c) cooling the hot crude solution of (K) to a temperature of about 20° C. provide a cooled (K) slurry;
(2-d) adding a solvent comprising methyl tert-butyl ether (MTBE) to the cooled (K) slurry to provide a purified (K) slurry; and
(2-e) filtering the purified (K) slurry to provide (K).

9. (canceled)

10. The method of claim 1, wherein (I) is prepared by reacting 2-methoxy-4-(B(OR^{1a})(OR^{1b}))-benzaldehyde (H): 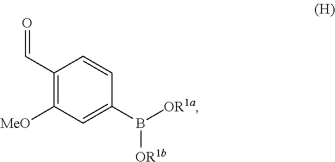
and 6-(2-chloro-3-(2,3-dichloropyridin-4-yl)phenyl)-2-methoxynicotinaldehyde (F): 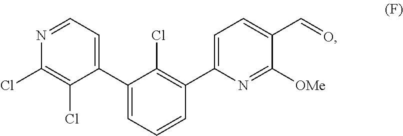 [SMILES: COc1nc(-c2cccc(-c3ccnc(Cl)c3Cl)c2Cl)ccc1C=O]
in the presence of a palladium catalyst and a base;
wherein each R^{1a} and R^{1b} are each independently selected from the group consisting of C₁-C₆ alkyl and C₃-C₈ cycloalkyl,
or R^{1a} and R^{1b} may combine with the atoms to which they are bound to form a C₂-C₃ heterocycloalkyl.

11. The method of claim 10, wherein at least one of the following applies:
(a) the palladium catalyst comprises Pd(PPh₃)₄;
(b) the palladium catalyst is present in an amount ranging from about 0.1 mol % to about 5 mol %;
(c) the base comprises K₂CO₃;
(d) the reaction of (H) and (F) occurs in the presence of a solvent, optionally wherein the solvent comprises at least one of 2-methyltetrahydrofuran (MeTHF), dimethylformamide (DMF), and water;
(f) the reaction of (H) and (F) occurs at a temperature of about 65° C. to about 70° C.; and
(g) purification of (I) comprises adding a N-acetyl cysteine to the reaction of (H) and (F).

12-17. (canceled)

18. The method of claim 10, wherein (H) is prepared by reacting 4-Z²-2-methoxybenzaldehyde (G): 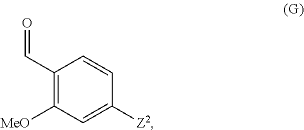
wherein Z² is selected from the group consisting of Cl, Br, and I;
and a borylating reagent in the presence of a palladium catalyst and a base.

19. The method of claim 18, wherein at least one of the following applies:
(a) the borylating reagent comprises bis(pinacolato)diboron;
(b) the palladium catalyst comprises Pd(dppf)Cl₂;
(c) the base comprises KOAc;
(d) the reaction of (G) and the borylating reagent occurs in the presence of a solvent, optionally wherein the solvent comprises dimethylformamide (DMF) or toluene;
(e) the reaction of (G) and the borylating reagent occurs at a temperature of about 80° C. to about 90° C.; and
(f) purification of (H) comprises at least one of (i) steps (3-a) to (3-b) and (ii) steps (4-a) to (4-d):
(3-a) adding N-acetyl cysteine to the reaction of (G) and the borylating reagent; and
(3-b) adding activated carbon to the reaction of (G) and the borylating reagent; and
(4-a) providing crude (H) in a solvent comprising 2-propanol to afford a dilute crude solution of (H),
wherein the dilute crude solution of (H) has a concentration of about 390 g/L to about 430 g/L;
(4-b) at least partially evaporating the dilute crude solution of (H) to provide a concentrated crude solution of (H), wherein the at least partial evaporation optionally comprises heating the dilute crude solution of (H) to a temperature of about 50° C., and further optionally maintaining the temperature for a period of about 1.5 h;
(4-c) cooling the concentrated crude solution of (H) to a temperature of about 6° C., provide a purified (H) slurry, wherein the cooling optionally occurs over a period of about 3 h; and
(4-d) filtering the purified (H) slurry to provide (H).

20-27. (canceled)

28. The method of claim 10, wherein (F) is prepared by reacting 6-(3-Z¹-2-chlorophenyl)-2-methoxynicotinaldehyde (D): 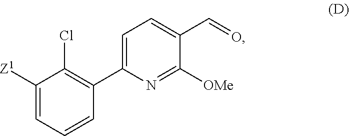
wherein Z¹ is selected from the group consisting of Br and I;
and 2,3-dichloro-4-(B(OR^{2a})(OR^{2b}))-pyridine (E): 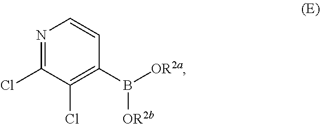
in the presence of a palladium catalyst and a base;
wherein each R^{2a} and R^{2b} are each independently selected from the group consisting of C₁-C₆ alkyl and C₃-C₈ cycloalkyl,
or R^{2a} and R^{2b} may combine with the atoms to which they are bound to form a C₂-C₃ heterocycloalkyl.

29. The method of claim 28, wherein at least one of the following applies:
(a) R^{2a} and R^{2b} are each independently H;
(b) the palladium catalyst comprises Pd(amphos)Cl₂;
(c) the palladium catalyst is present in an amount ranging from about 0.1 mol % to about 5 mol %;
(d) the base comprises K₂HPO₄;
(e) the reaction of (D) and (E) comprises addition of (E) to a reaction vessel comprising (D), wherein (E) is optionally added to the vessel comprising (D) over a period of about 3.5 h;
(f) the reaction of (D) and (E) occurs in the presence of a solvent, optionally wherein the solvent comprises 2-methyltetrahydrofuran (MeTHF), dimethylacetamide (DMAc), and/or water;
(g) the reaction of (D) and (E) occurs at a temperature of about 65° C. to about 70° C.;
(h) purification of (F) comprises adding a N-acetyl cysteine to the reaction of (D) and (E); and
(i) purification of (F) comprises:
(5-a) providing crude (F) in a solvent comprising 2-propanol to afford a dilute crude solution of (F),
wherein the dilute crude solution of (F) has a concentration of about 100 g/L to about 150 g/L;
(5-b) at least partially evaporating the dilute crude solution of (F) to provide a concentrated crude solution of (F), wherein the at least partial evaporation optionally comprises heating the dilute crude solution of (F) to a temperature of about 40° C. to about 55° C., and further optionally maintaining the temperature for a period of about 1 h;
(5-c) cooling the concentrated crude solution of (F) to a temperature of about 20° C. to about 25° C., to provide a crude (F) slurry, wherein the cooling optionally occurs over a period of about 3 h; and
(5-d) filtering the crude (F) slurry to provide (F), wherein the filtering optionally further comprises washing with a solvent comprising 2-propanol.

30-38. (canceled)

39. The method of claim 10, wherein (F) is prepared by reacting 6-(2-chloro-3-(B(OR^{3a})(OR^{3b}))phenyl)-2-methoxynicotinaldehyde (D′): 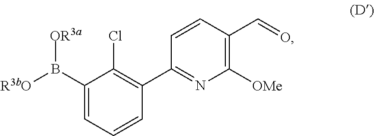
and 2,3-dichloro-4-Z¹-pyridine (E′): 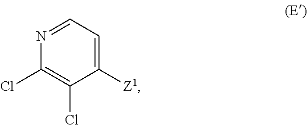
wherein Z¹ is selected from the group consisting of Br an I;
in the presence of a palladium catalyst and a base;
wherein each R^{3a} and R^{3b} are each independently selected from the group consisting of C₁-C₆ alkyl and C₃-C₈ cycloalkyl,
or R^{3a} and R^{3b} may combine with the atoms to which they are bound to form a C₂-C₃ heterocycloalkyl.

40. The method of claim 39, wherein at least one of the following applies:
(a) (D′) is 6-(2-chloro-3-(4,4,5,5-tetramethyl-1,3,2-dioxaborolan-2-yl)phenyl)-2-methoxynicotinaldehyde;
(b) the palladium catalyst comprises Pd(dppf)Cl₂;
(c) the palladium catalyst is present in an amount ranging from about 0.1 mol % to about 5 mol %;
(d) the base comprises K₂CO₃;
(e) the reaction of (D′) and (E′) occurs in the presence of a solvent, optionally wherein the solvent comprises 2-methyltetrahydrofuran (MeTHF) and water;
(f) the reaction of (D′) and (E′) occurs at a temperature of about 55° C.;
(g) purification of (F) comprises adding N-acetyl cysteine to the reaction of (D′) and (E′) or (6-a) to (6-d):
(6-a) providing crude (F) in 2-propanol to afford a dilute crude solution of (F), wherein the dilute crude solution of (F) has a concentration of about 100 g/L to about 140 g/L;
(6-b) at least partially evaporating the dilute crude solution of (F) to provide a concentrated crude solution of (F), wherein the at least partial evaporation optionally comprises heating the dilute crude solution of (F) to a temperature of about 50° C. to about 55° C., and further optionally maintaining the temperature for a period of about 1 h;
(6-c) cooling the concentrated crude solution of (F) to a temperature of about 20° C. to about 25° C., to provide a purified (F) slurry; and
(6-d) filtering the purified (F) slurry to provide (F).

41-48. (canceled)

49. The method of claim 39, wherein (D′) is prepared by reacting (D): 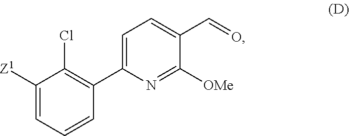
wherein Z¹ is selected from the group consisting of Br and I;
and a borylating reagent in the presence of a palladium catalyst and a base.

50. The method of claim 49, wherein at least one of the following applies:
(a) the borylating reagent comprises bis(pinacolato)diboron;
(b) the palladium catalyst comprises Pd(dppf)Cl₂;
(c) the palladium catalyst is present in an amount ranging from about 0.1 mol % to about 7 mol %;
(d) wherein the base comprises KOAc;
(e) the reaction of (D) and the borylating reagent occurs in the presence of a solvent, optionally wherein the solvent comprises dimethylformamide (DMF) and/or toluene;
(f) the reaction of (D) and the borylating reagent occurs at temperature of about 90° C. to about 100° C.;
(g) purification of (D′) comprises at least one of (i) steps (7-a) to (7-b) and (ii) steps (8-a) to (8-c):
(7-a) adding a N-acetyl cysteine to the reaction of (D) and the borylating reagent; and
(7-b) adding activated carbon to the reaction of (D) and the borylating reagent; and
(8-a) providing crude (D′) in a solvent comprising 2-propanol to afford a crude solution of (D′),
wherein the crude solution of (D′) has a concentration of about 140 g/L to about 180 g/L;
(8-b) cooling the crude solution of (D′) to a temperature of about 0° C. to about 5° C., to provide a purified (D′) slurry; and
(8-c) filtering the purified (D′) slurry to provide (D′).

51-58. (canceled)

59. The method of claim 28, wherein (D) is prepared by reacting 1-(B(OR^{4a})(OR^{4b}))-2-chloro-3-Z¹-benzene (B): 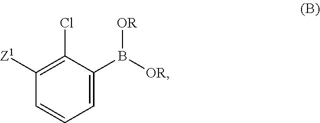
and 6-Z²-2-methoxynicotinaldehyde (C): 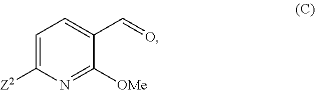
in the presence of a palladium catalyst and a base;
wherein Z¹ is selected from the group consisting of Br and I;
wherein Z² is selected from the group consisting of Cl, Br, and I;
wherein each R^{4a} and R^{4b} are each independently selected from the group consisting of C₁-C₆ alkyl and C₃-C₈ cycloalkyl,
or R^{4a} and R^{4b} may combine with the atoms to which they are bound to form a C₂-C₃ heterocycloalkyl.

60. The method of claim 59, wherein at least one of the following applies:
(a) (B) is 2-(3-bromo-2-chlorophenyl)-4,4,5,5-tetramethyl-1,3,2-dioxaborolane;
(b) the palladium catalyst comprises Pd(PPh₃)₄;
(c) the base comprises K₂CO₃;
(d) the reaction of (B) and (C) occurs in the presence of a solvent, optionally wherein the solvent comprises 2-methyltetrahydrofuran (MeTHF) and/or water;
(e) the reaction of (B) and (C) occurs at a temperature of about 50° C. to about 60° C.;
(f) purification of (D) comprises adding N-acetyl cysteine to the reaction of (B) and (C) or steps (9-a) to (9-d):
(9-a) providing crude (D) in solvent comprising 2-propanol to afford a dilute crude solution of (D),
wherein the dilute crude solution of (D) has a concentration of about 160 g/L to about 220 g/L;
(9-b) at least partially evaporating the dilute crude solution of (D) to provide a concentrated crude solution of (D), wherein the at least partial evaporation optionally comprises heating the dilute crude solution of (D) to a temperature of about 50° C. to about 55° C., and further optionally maintaining the temperature for a period of about 1 h;
(9-c) cooling the concentrated crude solution of (D) to a temperature of about 20° C. to about 25° C., to provide a purified (D) slurry; and
(9-d) filtering the purified (D) slurry to provide (D).

61-68. (canceled)

69. The method of claim 59, wherein (B) is prepared by:
(a) reacting 1-Z¹-2-chloro-3-Z¹-benzene (A): 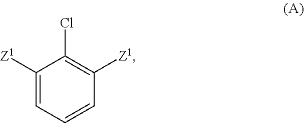
wherein each occurrence of Z¹ is independently selected from the group consisting of Br and I;
and an organomagnesium halide to form an arylmagnesium intermediate; and
(b) reacting the arylmagnesium intermediate with a borate.

70. The method of claim 69, wherein the borate is: 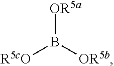
wherein each R^{5a}, R^{5b}, and R^{5c} are each independently selected from the group consisting of C₁-C₆ alkyl and C₃-C₈ cycloalkyl,
or any two selected from the group consisting of R^{5a}, R^{5b}, and R^{5c} may combine with the atoms to which they are bound to form a C₂-C₃ heterocycloalkyl.

71. The method of claim 69, wherein at least one of the following applies:
(a) the borate comprises 2-isopropoxy-4,4,5,5-tetramethyl-1,3,2-dioxaborolane;
(b) the organomagnesium halide comprises i-PrMgCl, wherein the i-PrMgCl is optionally a solution comprising i-PrMgCl complexed with LiCl (i.e., an i-PrMgCl·LiCl solution);
(c) the reaction of (A) and the organomagnesium halide occurs in a solvent, optionally wherein the solvent comprises 2-methyltetrahydrofuran (MeTHF);
(d) the reaction of (A) and the organomagnesium halide occurs at a temperature of about −25° C. to about −15° C.; and
(e) the reaction of the arylmagnesium intermediate and the borate occurs at a temperature of about −18° C. to about −15° C., wherein the reaction is optionally subsequently warmed to a temperature of about 2° C.

72-76. (canceled)

77. The method of claim 28, wherein (D) is prepared by:
(a) providing 1-Z¹-2-chloro-3-Z¹-benzene (A): 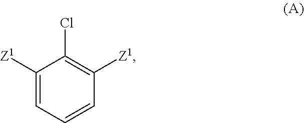
wherein each occurrence of Z¹ is independently selected from the group consisting of Br and I;
and an organomagnesium halide to form an arylmagnesium intermediate;
(b) reacting the arylmagnesium intermediate with a borate to provide a boronic ester intermediate; and
(c) reacting the boronic ester intermediate and 6-Z²-2-methoxynicotinaldehyde (C): 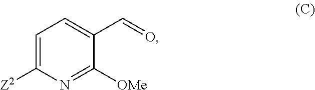
wherein Z² is selected from the group consisting of Cl, Br, and I;
in the presence of a palladium catalyst and a base.

78. The method of claim 77, wherein at least one of the following applies:
(a) the organomagnesium halide comprises i-PrMgCl, wherein the i-PrMgCl is optionally a solution comprising i-PrMgCl complexed with LiCl (i.e., an i-PrMgCl·LiCl solution),
(b) the reaction of (A) and the organomagnesium halide occurs in a solvent, optionally wherein the solvent comprises 2-methyltetrahydrofuran (MeTHF);
(c) the reaction of (A) and the organomagnesium halide occurs at a temperature of about −25° C. to about −15° C.; and
(d) the reaction of the arylmagnesium intermediate and the borate occurs at a temperature of about −18° C. to about −15° C., wherein the reaction is optionally subsequently warmed to a temperature of about 2° C.;
(e) the borate comprises 2-isopropoxy-4,4,5,5-tetramethyl-1,3,2-dioxaborolane;
(f) the palladium catalyst comprises Pd(PPh₃)₄;
(g) the palladium catalyst is present in an amount ranging from about 0.1 mol % to about 5 mol %;
(h) the base comprises K₂CO₃;
(i) the reaction of the boronic ester intermediate and (C) occurs in the presence of a solvent, optionally wherein the solvent comprises 2-methyltetrahydrofuran (MeTHF) and/or water;
(i) the reaction of the boronic ester intermediate and (C) occurs at a temperature of about 50° C. to about 60° C.; and
(k) purification of (D) comprises adding N-acetyl cysteine to the reaction of (C) and the boronic ester intermediate or steps (10-a) to (10-d):
(a) providing crude (D) in a solvent comprising 2-propanol to afford a dilute crude solution of (D),
wherein the dilute crude solution of (D) has a concentration of about 160 g/L to about 220 g/L;
(b) at least partially evaporating the dilute crude solution of (D) to provide a concentrated crude solution of (D), wherein the at least partial evaporation optionally comprises heating the dilute crude solution of (D) to a temperature of about 50° C. to about 55° C., and further optionally maintaining the temperature for a period of about 1 h;
(c) cooling the concentrated crude solution of (D) to a temperature of about 20° C. to about 25° C., to provide a purified (D) slurry; and
(d) filtering the purified (D) slurry to provide (D).

79-82. (canceled)

83. The method of claim 77, wherein the borate is: 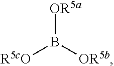
wherein each R^{5a}, R^{5b}, and R^{5c} are each independently selected from the group consisting of C₁-C₆ alkyl and C₃-C₈ cycloalkyl,
or any two selected from the group consisting of R^{5a}, R^{5b}, and R^{5c} may combine with the atoms to which they are bound to form a C₂-C₃ heterocycloalkyl.

84-92. (canceled)
